(19) United States
(12) Patent Application Publication
(10) Pub. No.: US 2025/0345452 A1
(43) Pub. Date: Nov. 13, 2025

(54) METHODS FOR THE TREATMENT OF PATIENTS WITH FOLATE RECEPTOR ALPHA-EXPRESSING CANCERS

(71) Applicant: ImmunoGen Switzerland GmbH, Zug (CH)

(72) Inventors: Eric Westin, Concord, MA (US); Michael Method, Martinsville, IN (US); Yuemei Wang, Sudbury, MA (US)

(21) Appl. No.: 19/194,134

(22) Filed: Apr. 30, 2025

Related U.S. Application Data
(60) Provisional application No. 63/641,371, filed on May 1, 2024.
(60) Provisional application No. 63/679,384, filed on Aug. 5, 2024.

(51) Int. Cl. A61K 47/68; A61P 35/00

(52) U.S. Cl. CPC A61K 47/6869; A61K 47/68033; A61P 35/00

(57) ABSTRACT

The present disclosure provides methods of treating patients with cancers that express folate receptor alpha (FRα), e.g., a recurrent platinum-sensitive ovarian cancer (rPSOC), comprising administering to the patient an anti-FRα immunoconjugate.

### CROSS-REFERENCE TO RELATED APPLICATIONS

This application claims the benefit of U.S. application No. 63/641,371, filed May 1, 2024, and U.S. application No. 63/679,384, filed Aug. 5, 2024, each of which is incorporated herein by reference in its entirety.

### FIELD

The disclosure relates to methods of treating patients with cancers that express folate receptor alpha (FRα).

### REFERENCE TO SEQUENCE LISTING SUBMITTED ELECTRONICALLY

The content of the electronically submitted sequence listing (Name: 6776_6602_Sequence_Listing.xml; Size: 24,870 bytes; and Date of Creation: Apr. 23, 2025) filed with the application is incorporated herein by reference in its entirety.

### BACKGROUND

Epithelial ovarian cancer (EOC; includes ovarian, fallopian tube, and primary peritoneal cancer), is the most lethal gynecologic malignancy, with a 5-year relative survival rate of approximately 51%. National Cancer Institute Surveillance, Epidemiology, and End Results Program. Cancer Stat Facts: Ovarian Cancer. Accessed Apr. 4, 2023. seer.cancer.gov/statfacts/html/ovary.html; Kuroki & Guntupalli, BMJ 371:m3773, 2020. In 2022, approximately 325,000 new EOC cases and 207,000 EOC-related deaths occurred globally. International Agency for Research on Cancer; World Health Organization. Ovary. Accessed Apr. 15, 2024. gco.iarc.who.int/media/globocan/factsheets/cancers/25-ovary-fact-sheet.pdf. Most patients are diagnosed with advanced-stage disease, and for these patients, first-line therapy typically consists of primary cytoreductive surgery and platinum-based chemotherapy, followed by observation or maintenance treatment with bevacizumab, a poly(adenosine diphosphate [ADP]-ribose) polymerase inhibitor (PARPi), or combination of bevacizumab plus PARPi.1,2 Although initial platinum-based chemotherapy yields high response rates, most patients will experience recurrence. Chien et al, Front Oncol 3:251, 2013.

Treatment for recurrent EOC is often based on platinum-free interval (PFI), or the time between recurrence and last platinum-based therapy. Salani et al, Gynecol Oncol 146:3-10, 2017; Luvero et al, Ther Adv Med Oncol 6:229-39, 2014. In patients with a PFI>6 months (traditionally referred to as platinum-sensitive ovarian cancer [PSOC]), the standard of care includes re-treatment with platinum-based chemotherapy—which typically consists of a platinum-based doublet with or without bevacizumab at first recurrence—followed by maintenance therapies (e.g., PARPi or bevacizumab) in select circumstances or by observation. Kuroki & Guntupalli BMJ 371:m3773, 2020. These maintenance therapies may yield additive benefits in patients who have responded to platinum-based chemotherapy. Arnaoutoglou et al, Medicina (Kaunas) 59:1183, 2023, Gupta et al, J Ovarian Res 12:103, 2019. However, after each successive disease recurrence, the efficacy of chemotherapy diminishes (i.e., first-line objective response rate [ORR] of 92% versus fifth-line ORR of 17%), as does a patient's ability to tolerate the accompanying cumulative toxicities associated with treatment, increased risk of platinum hypersensitivity reactions, and concern for cross-resistance to platinum reinduction after PARPi maintenance. Kessous et al, Int J Cancer 148:2304-2312, 2021. Further, among patients with later-line PSOC deemed unsuitable for platinum-based chemotherapy, single-agent non-platinum chemotherapies—such as etoposide, topotecan, and pegylated liposomal doxorubicin—have demonstrated modest ORRs in PARPi-naïve patients, ranging from 19% to 34%. Ozols RF: Oral etoposide for the treatment of recurrent ovarian cancer. Drugs 58 Suppl 3:43-9, 1999; Morris et al, Gynecol Oncol 109:346-52, 2008; ten Bokkel Huinink et al, J Clin Oncol 15:2183-93, 1997; and Monk et al, J Clin Oncol 28:3107-14, 2010.

The mechanisms of resistance to platinum-based chemotherapy and PARPi are complex and multifactorial, with overlapping mechanisms of acquired resistance. McMullen et al, Cancers (Basel) 12, 2020. This is clinically relevant given the significant use of PARPi therapy in patients with EOC; approximately 87% of patients with EOC in the US were eligible for PARPi treatment in 2023. Carballo et al, Gynecol Oncol 187:204-211, 2024. Several reports have suggested that patients with disease progression on or after PARPi exhibit diminished response to subsequent platinum-based treatments. O'Malley et al, Target Oncol 18:471-503, 2023; Frenel et al, Ann Oncol 33:1021-1028, 2022; Cecere et al, Gynecol Oncol 156:38-44, 2020; Romeo et al, Cancers (Basel) 14, 2022; Harter P, et al. Presented at: 2023 American Society of Clinical Oncology; Jun. 2-6, 2023; Chicago, IL. Abstract 5550; Rose et al, Anticancer Drugs 32:1086-1092, 2021; Park et al, Gynecol Oncol 165:97-104, 2022. Collectively, these findings highlight the critical need to identify effective and tolerable therapies for patients with PSOC, especially those who have received a PARPi and subsequently experienced disease progression.

Folate receptor-α (FRα or FOLR1) is a glycosylphosphatidylinositol-linked cell-surface glycoprotein that has high affinity for folates. Its physiologic role in normal and cancerous tissues has not yet been fully elucidated. Most normal tissues do not express FRα, and transport of physiologic folates into most cells is thought to be mediated by several other proteins, most notably, reduced folate carrier. High levels of FRα have been found in serous and endometrioid epithelial ovarian cancer, endometrial adenocarcinoma, and non-small cell lung cancer of the adenocarcinoma subtype. Importantly, FRα expression is maintained in metastatic foci and recurrent carcinomas in ovarian cancer patients, and after chemotherapy in epithelial ovarian and endometrial cancers. These properties, together with the highly restricted expression of FRα on normal tissues, make FRα a highly promising target for targeted therapies such as ADCs.

Mirvetuximab soravtansine (IMGN853), a folate targeting antibody drug conjugate (ADC) that comprises a FRα targeting antibody conjugated to a potent tubulin-acting maytansinoid, DM4, was recently evaluated in the clinic in platinum-resistant ovarian cancer patients with recurrent platinum-sensitive, high-grade serous epithelial ovarian cancer (EOC), primary peritoneal, or fallopian tube cancer whose tumors express a high-level of FRα as measured by immunohistochemistry (IHC) staining. Such cancers are referred to as rPSOCs (recurrent platinum-sensitive ovarian cancers). Ovarian, peritoneal, and fallopian tube cancers are not distinct entities, but represent a spectrum of diagnoses that originate in the Mullerian tissue. Primary fallopian tube carcinoma and peritoneal cancers are considered part of EOC with the same treatment and outcomes. IMGN853 received full US FDA approval in March 2024 for patients with FRα-positive platinum-resistant ovarian cancer (PROC) with 1-3 prior lines of therapy. For patients with FRα-expressing ovarian, fallopian tube, or primary peritoneal cancer, the NCCN Clinical Practice Guidelines in Oncology (NCCN Guidelines®) recommend IMGN853 monotherapy as a preferred targeted therapy option in platinum-resistant disease (category 1); the combination of IMGN853 plus bevacizumab is also recommended as useful in certain circumstances in platinum-resistant disease (category 2A) and platinum-sensitive disease (category 2B). NCCN Treatment Guidelines, v2 2024.

The PICCOLO Phase 2 trial was designed to evaluate the efficacy and safety of IMGN853 in patients with rPSOC, primary peritoneal, or fallopian tube cancer, whose tumors express a high level of FRα, as a potential improvement over the overall response rate (ORR) of patients that have had at least two lines of prior therapy. This included at least two lines of platinum-containing therapy or one line with a documented platinum allergy. While platinum remains the most active treatment for earlier lines of ovarian cancer, the benefit of retreatment with platinum is generally less with each subsequent line of therapy. The dearth of efficacious treatment options beyond the second line of therapy for patients with PSOC represents a considerable unmet need. Kessous et al, Int J Cancer 148:2304-2312, 2021; O'Malley et al, Target Oncol 18:471-503, 2023; in Lele S (ed): Ovarian Cancer. Brisbane (AU), 2022. Therefore, there is a need for an improvement in third-line treatment options for patients with rPSOC, primary peritoneal, or fallopian tube cancer, whose tumors express a high level of FRα.

### SUMMARY

Provided herein are methods of treating a human patient having a folate receptor alpha (FRα)-expressing recurrent platinum-sensitive ovarian cancer (rPSOC).

In some aspects, the disclosure provides a method for treating a folate receptor alpha (FRα)-expressing recurrent platinum-sensitive ovarian cancer (rPSOC) in a human patient in need thereof, comprising intravenously administering once every three weeks to the patient a therapeutically effective amount of mirvetuximab soravtansine, wherein the therapeutically effective amount is 6 mg/kg of adjusted ideal body weight (AIBW) of the patient. In some aspects, the administration increases the human patient's overall survival, the administration results in a decrease in tumor size, the administration results in a decrease in cancer antigen 125 (CA-125) expression, the administration results in an objective response rate (ORR) of about greater than 25%, wherein the administration results in the human patient achieving a duration of response (DOR) of at least 4 months, the administration results in the human patient achieving a progression-free survival (PFS) of at least 3.98 months, and/or the administration results in an about 80% chance of the human patient achieving a progression-free survival (PFS) of at least 3 months.

In some aspects, the rPSOC is epithelial ovarian cancer (EOC). In some aspects, the rPSOC is high-grade serous EOC. In some aspects, the rPSOC is primary peritoneal cancer. In some aspects, the rPSOC is fallopian tube cancer.

In some aspects of the methods disclosed herein, the patient has had at least one line of platinum-containing therapy prior to initiation of the present method. In some aspects, the patient has had a platinum-free interval of at least 6 months prior to initiation of the present method. In some aspects, the patient has had a platinum-free interval of between 6 months and 12 months prior to initiation of the present method. In some aspects, the patient has had a platinum-free interval of more than 12 months prior to initiation of the present method. In some aspects, the patient has had one independent non-platinum cytotoxic therapy prior to initiation of the present method. In some aspects, the patient has a platinum allergy. In some aspects, the patient has had at least two lines of therapy prior to initiation of the present method. In some aspects, the mirvetuximab soravtansine is administered at a rate of 1 mg/min. In some aspects, the mirvetuximab soravtansine is administered at a rate of 3 mg/min. In some aspects, the mirvetuximab soravtansine is administered at a rate of 5 mg/min. In some aspects, the mirvetuximab soravtansine is administered as a monotherapy.

In some aspects of the methods disclosed herein, the administration of the mirvetuximab soravtansine increases the patient's overall survival.

In some aspects of the methods disclosed herein, a cancer sample from the patient exhibits increased expression of FRα measured by an immunohistochemistry (IHC) assay that can determine the percentage of cancer cells with moderate (2+) and/or strong (3+) membrane staining compared to the total number of viable tumor cells. In some aspects, the cancer sample is a formalin fixed paraffin embedded sample. In some aspects, at least 25% of cells in a cancer sample obtained from the patient have an immunohistochemistry (IHC) score of at least 2. In some aspects, at least 75% of cells in a cancer sample obtained from the patient have an immunohistochemistry (IHC) score of at least 2. In some aspects, a positive FRα expression score is defined as ≥75% of viable tumor cells with moderate (2+) and/or strong (3+) membrane staining. In some aspects, a negative FRα expression is defined as <75% of viable tumor cells with moderate (2+) and/or strong (3+) membrane staining. In some aspects, the patient's FRα-expressing cancer sample displays a positive FRα expression score as defined by the assay.

In some aspects of the methods disclosed herein, the subject has at least one measurable disease lesion as defined by RECIST v1.1.

In some aspects of the methods disclosed herein, the patient has a BRCA mutation.

In some aspects of the methods disclosed herein, the patient does not have a BRCA mutation.

In some aspects of the methods disclosed herein, the patient has had PARPi therapy prior to initiation of the present method. In some aspects, the patient has had disease progression (PD) while receiving PARPi therapy or within 30 days of receiving PARPi prior to initiation of the present method. In some aspects, the patient has had no disease progression (PD) while receiving PARPi therapy or within 30 days of receiving PARPi prior to initiation of the present method.

In some aspects of the methods disclosed herein, the patient has had bevacizumab therapy prior to initiation of the present method. In some aspects, the patient has had at least one line of therapy comprising bevacizumab prior to initiation of the present method. In some aspects, the patient has had more than one line of therapy comprising bevacizumab prior to initiation of the present method.

In some aspects of the methods disclosed herein, the patient has had taxane therapy prior to initiation of the present method. In some aspects, the patient has had at least one line of therapy comprising taxane prior to initiation of the present method. In some aspects, the patient has had more than one line of therapy comprising taxane prior to initiation of the present method.

In some aspects, the methods disclosed herein further comprise administering a corticosteroid eye drop to the patient. In some aspects, the steroid comprises 1% prednisolone or difluprednate 0.05%.

In some aspects, the methods disclosed herein further comprise administering to the subject artificial tears.

In some aspects of the methods disclosed herein, the administration results in a decrease in tumor size. In some aspects, the administration results in a decrease in cancer antigen 125 (CA-125).

In some aspects of the methods disclosed herein, the administration results in an objective response rate (ORR) of about greater than 25%.

In some aspects of the methods disclosed herein, the administration results in an duration of response (DOR) of at least 4 months.

In some aspects of the methods disclosed herein, administration of the immunoconjugate to the results in a progression-free survival (PFS) of at least 3.98 months.

In some aspects of the methods disclosed herein, administration of the mirvetuximab soravtansine increases the patient's overall survival.

In some embodiments, the disclosure provides:
[1.] A method for treating a folate receptor alpha (FRα)-expressing recurrent platinum-sensitive ovarian cancer (rPSOC) in a human patient in need thereof, comprising intravenously administering once every three weeks to the patient a therapeutically effective amount of mirvetuximab soravtansine,
[wherein the therapeutically effective amount is 6 mg/kg of adjusted ideal body weight (AIBW) of the patient.
[2.] A method for treating a folate receptor alpha (FRα)-expressing recurrent platinum-sensitive ovarian cancer (rPSOC) in a human patient in need thereof, comprising intravenously administering once every three weeks to the patient a therapeutically amount of mirvetuximab soravtansine, wherein the therapeutically effective amount is 6 mg/kg of adjusted ideal body weight (AIBW) of the patient, and wherein
   the administration increases the human patient's overall survival,
   the administration results in a decrease in tumor size,
   the administration results in a decrease in cancer antigen 125 (CA-125) expression,
   the administration results in an objective response rate (ORR) of about greater than 25%,
   wherein the administration results in the human patient achieving a duration of response (DOR) of at least 4 months,
   the administration results in the human patient achieving a progression-free survival (PFS) of at least 3.98 months, and/or
   the administration results in an about 80% chance of the human patient achieving a progression-free survival (PFS) of at least 3 months.
[3.] The method of [1] or [2], wherein the rPSOC is epithelial ovarian cancer (EOC).
[4.] The method of [2], wherein the rPSOC is high-grade serous EOC.
[5.] The method of [1] or [2], wherein the rPSOC is primary peritoneal cancer.
[6.] The method of [1] or [2], wherein the rPSOC is fallopian tube cancer.
[7.] The method of any one of [1]-[6], wherein the patient has had at least one line of platinum-containing therapy prior to initiation of the present method.
[8.] The method of [7], wherein the patient has had a platinum-free interval of at least 6 months prior to initiation of the present method.
[9.] The method of [7], wherein the patient has had a platinum-free interval of between 6 months and 12 months prior to initiation of the present method.
[10.] The method of [7], wherein the patient has had a platinum-free interval of more than 12 months prior to initiation of the present method.
[11. The method of any one of [1]-[10], wherein the patient has had one independent non-platinum cytotoxic therapy prior to initiation of the present method.
[12.] The method of any one of [1]-[11], wherein the patient has a platinum allergy.
[13.] The method of any one of [1]-[11], wherein the patient has had at least two lines of therapy prior to initiation of the present method.
[14.] The method of any one of [1]-[13], wherein the mirvetuximab soravtansine is administered at a rate of 1 mg/min.
[15.] The method of any one of [1]-[13], wherein the mirvetuximab soravtansine is administered at a rate of 3 mg/min.
[16.] The method of any one of [1]-[13], wherein the mirvetuximab soravtansine is administered at a rate of 5 mg/min.
[17.] The method of [1]-[13], wherein the mirvetuximab soravtansine is administered as a monotherapy.
[18.] The method of any one of [1]-[17], wherein a cancer sample from the patient exhibits increased expression of FRα measured by an immunohistochemistry (IHC) assay that can determine the percentage of cancer cells with moderate (2+) and/or strong (3+) membrane staining compared to the total number of viable tumor cells.
[19.] The method of [18], wherein the cancer sample is a formalin fixed paraffin embedded sample.
[20.] The method of [18] or [19], wherein at least 25% of cells in a cancer sample obtained from the patient have an immunohistochemistry (IHC) score of at least 2.
[21.] The method of any one of [18]-[20], wherein at least 75% of cells in a cancer sample obtained from the patient have an immunohistochemistry (IHC) score of at least 2.
[22.] The method of any one of [18]-[21], wherein a positive FRα expression score is defined as ≥75% of viable tumor cells with moderate (2+) and/or strong (3+) membrane staining.
[23.] The method of any one of [18]-[21], wherein a negative FRα expression is defined as <75% of viable tumor cells with moderate (2+) and/or strong (3+) membrane staining.
[24.] The method of any of [18]-[23], wherein the patient's FRα-expressing cancer sample displays a positive FRα expression score as defined by the assay.
[25.] The method of any one of [1]-[24], wherein the subject has at least one measurable disease lesion as defined by RECIST v1.1.
[26.] The method of any one of [1]-[25], wherein the patient has a BRCA mutation.
[27.] The method of any one of [1]-[26], wherein the patient does not have a BRCA mutation.
[28.] The method of any one of [1]-[27], wherein the patient has had PARPi therapy prior to initiation of the present method.
[29.] The method of [28], wherein the patient has had disease progression (PD) while receiving PARPi therapy or within 30 days of receiving PARPi prior to initiation of the present method.
[30.] The method of [28], wherein the patient has had no disease progression (PD) while receiving PARPi therapy or within 30 days of receiving PARPi prior to initiation of the present method.
[31.] The method of any one of [1]-[30], wherein the patient has had bevacizumab therapy prior to initiation of the present method.
[32.] The method of any one of [1]-[31], wherein the patient has had taxane therapy prior to initiation of the present method.
[33.] The method of any one of [1]-[32], further comprising administering a corticosteroid eye drop to the patient.
[34.] The method of [33], wherein the steroid comprises 1% prednisolone or difluprednate 0.05%.
[35.] The method of any one of [1]-[34], further comprising administering to the subject artificial tears.

### DETAILED DESCRIPTION

### I. Definitions

To facilitate an understanding of the present disclosure, a number of terms and phrases are defined below.

The terms “folate receptor 1,” “FRα,” “folate receptor alpha (FR-α),” or “FOLR1” as used herein, refers to any native FRα polypeptide, unless otherwise indicated. The terms encompasses “full-length,” unprocessed FRα polypeptide as well as any form of FRα polypeptide that results from processing within the cell. The term also encompasses naturally occurring variants of FRα, e.g., those encoded by splice variants and allelic variants. The FRα polypeptides described herein can be isolated from a variety of sources, such as from human tissue types or from another source, or prepared by recombinant or synthetic methods. Where specifically indicated, “FRα” can be used to refer to a nucleic acid that encodes a FRα polypeptide. Human FRα sequences are known and include, for example, the sequences publicly available at UniProtKB Accession No. P15328 (including isoforms). FRα can include a signal peptide (amino acids 1-24), the FRα protein chain (amino acids 25-234), and a propeptide that can be cleaved (amino acids 235 to 257). As used herein, the term “full-length human FRα” refers to polypeptide comprising the amino acid sequence SEQ ID NO:1, and the term “mature human FRα” refers to a polypeptide comprising amino acids 25-234 of SEQ ID NO:1).

The term “anti-FRα antibody” or “an antibody that binds to FRα” refers to an antibody that is capable of binding FRα with sufficient affinity such that the antibody is useful as a diagnostic and/or therapeutic agent in targeting FRα. Unless otherwise specified, the extent of binding of an anti-FRα antibody to an unrelated, non-FRα protein is less than about 10% of the binding of the antibody to FRα as measured, e.g., by a radioimmunoassay (RIA). Examples of FRα antibodies are known in the art and are disclosed in U.S. Published Application Nos. 2012/0009181, 2012/0282175. and 2020/0362029, U.S. Pat. No. 9,200,073 B2, and PCT publication WO 2011/106528 A1, each of which is herein incorporated by reference in its entirety.

The huMov19 (M9346A) antibody is encoded by the plasmids deposited with the American Type Culture Collection (ATCC), located at 10801 University Boulevard, Manassas, VA 20110 on Apr. 7, 2010 under the terms of the Budapest Treaty and having ATCC deposit nos. PTA-10772 and PTA-10774. DM4 refers to N2′-deacetyl-N2′-(4-mercapto-4-methyl-1-oxopentyl) maytansinoid. “Sulfo-SPDB” refers to the N-succinimidyl 4-(2-pyridyldithio)-2-sulfobutanoate) linker.

The terms “high” FRα, “increased expression” of FRα, or “overexpression” of FRα in a particular tumor, tissue, or sample refers to FRα (a FRα polypeptide or a nucleic acid encoding such a polypeptide) that is present at a level higher than that which is present in a healthy or non-diseased (native, wild type) tissue, cells, or samples of the same type or origin. Such increased expression or overexpression can be caused, for example, by mutation, gene amplification, increased transcription, increased translation, or increased protein stability.

Membrane FRα expression can be measured by immunohistochemistry (IHC) and given a “staining intensity score” and/or a “staining uniformity score” by comparison to calibrated controls exhibiting defined scores (e.g., an intensity score of 3+ is given to the test sample if the intensity is comparable to the level 3+ calibrated control or an intensity of 2+ is given to the test sample if the intensity is comparable to the level 2+ calibrated control). A score of 0 refers to no staining. A score of 1+ refers to light (brown) staining. A score of 2+ refers to medium (brown) staining, and a score of 3+ refers to dark (brown) staining. Staining uniformity can be expressed as percentage (%) of cells staining at a certain intensity (e.g., 50% of cells staining at intensity of 1+, 2+, or 3+). With regard to membrane FRα expression by IHC, “PS” refers to percentage stained. Thus, for example, “≥75% of cells with PS2+ staining intensity” indicates that at least 75% of cells in sample have a staining intensity of at least 2+ (i.e., 2+ or 3+). Methods of measuring membrane FRα expression by IHC are disclosed, for example, in WO 2012/135675 and WO 2015/031815, each of which in herein incorporated by reference in its entirety.

A “reference sample” can be used to correlate and compare the results obtained with a test sample. Reference samples can be cells (e.g., cell lines, cell pellets), bodily fluids, or tissue. The FRα levels in the “reference sample” can be an absolute or relative amount, a range of amount, a minimum and/or maximum amount, a mean amount, and/or a median amount of FRα. A “reference sample” can also serve as a baseline of FRα expression to which the test sample is compared. The “reference sample” can include a prior sample or baseline sample from the same patient, a normal reference, or a reference from a relevant patient population. Generally, FRα levels are expressed as values in a standard curve. A standard curve is a quantitative method of plotting assay data to determine the concentration of FRα in a sample. In some aspects, a reference sample is an antigen standard comprising purified FRα or FRα-Fc. The methods of detection disclosed herein may involve a comparison between expression levels of FRα in a test sample and a “reference value” or “reference level.” In some aspects, the reference value is the expression level of the FRα in a reference sample. A reference value can be a predetermined value and can also be determined from reference samples (e.g., control biological samples) tested in parallel with the test samples. A reference value can be a single cut-off value, such as a median or mean or a range of values, such as a confidence interval. Reference values can be established for various subgroups of individuals, such as individuals predisposed to cancer, individuals having early or late stage cancer, male and/or female individuals, or individuals undergoing cancer therapy.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, chimeric antibodies, humanized antibodies, human antibodies, fusion proteins comprising an antibody, and any other modified immunoglobulin molecule so long as the antibodies exhibit the desired biological activity. As used herein, such antibodies include, for example, monospecific and bispecific (e.g., biparatopic) antibodies. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma. and mu, respectively. The different classes of immunoglobulins have different and well known subunit structures and three-dimensional configurations. Antibodies can be naked or conjugated to other molecules such as toxins (e.g., in an immunoconjugate), radioisotopes, etc.

The term “antibody fragment” or “antibody fragment thereof” refers to a portion of an intact antibody. An “antigen-binding fragment” refers to a portion of an intact antibody that binds to an antigen. An antigen-binding fragment can contain the antigenic determining variable regions of an intact antibody. Examples of antibody fragments include, but are not limited to Fab, Fab′, F(ab′)2, and Fv fragments, linear antibodies, and single chain antibodies (scFv). Antibody fragments can be naked or conjugated to other molecules such as toxins (e.g., in an immunoconjugate), radioisotopes, etc.

A “monoclonal” antibody or antigen-binding fragment thereof refers to a homogeneous antibody or antigen-binding fragment population involved in the highly specific recognition and binding of a single antigenic determinant, or epitope. This is in contrast to polyclonal antibodies that typically include different antibodies directed against different antigenic determinants. The term “monoclonal” antibody or antigen-binding fragment thereof encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal” antibody or antigen-binding fragment thereof refers to such antibodies and antigen-binding fragments thereof made in any number of manners including but not limited to by hybridoma, phage selection, recombinant expression, and transgenic animals.

The term “humanized” antibody or antigen-binding fragment thereof refers to forms of non-human (e.g. murine) antibodies or antigen-binding fragments that are specific immunoglobulin chains, chimeric immunoglobulins, or fragments thereof that contain minimal non-human (e.g., murine) sequences. Typically, humanized antibodies or antigen-binding fragments thereof are human immunoglobulins in which residues from the complementarity determining region (CDR) are replaced by residues from the CDR of a non-human species (e.g. mouse, rat, rabbit, hamster) that have the desired specificity, affinity, and capability (“CDR grafted”) (Jones et al., Nature 321:522-525 (1986); Riechmann et al., Nature 332:323-327 (1988); Verhoeyen et al., Science 239:1534-1536 (1988)). In some aspects, the Fv framework region (FR) residues of a human immunoglobulin are replaced with the corresponding residues in an antibody or fragment from a non-human species that has the desired specificity, affinity, and capability. The humanized antibody or antigen-binding fragment thereof can be further modified by the substitution of additional residues either in the Fv framework region and/or within the replaced non-human residues to refine and optimize antibody or antigen-binding fragment thereof specificity, affinity, and/or capability. In general, the humanized antibody or antigen-binding fragment thereof will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody or antigen-binding fragment thereof can also comprise at least a portion of an immunoglobulin constant region or domain (Fc), typically that of a human immunoglobulin. Examples of methods used to generate humanized antibodies are described in U.S. Pat. No. 5,225,539; Roguska et al., Proc. Natl. Acad. Sci., USA, 91(3):969-973 (1994), and Roguska et al., Protein Eng. 9(10):895-904 (1996). In some aspects, a “humanized antibody” is a resurfaced antibody.

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability (i.e., Kabat et al., Sequences of Proteins of Immunological Interest, (5th ed., 1991, National Institutes of Health, Bethesda Md.), “Kabat”); and (2) an approach based on crystallographic studies of antigen-antibody complexes (Al-lazikani et al, J. Molec. Biol. 273:927-948 (1997)). In addition, combinations of these two approaches are sometimes used in the art to determine CDRs.

A “constant” region of an antibody is not involved directly in binding an antibody to an antigen, but exhibits various effector functions, such as participation of the antibody in antibody-dependent cellular toxicity

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed., 1991, National Institutes of Health, Bethesda, Md.) (“Kabat”).

The amino acid position numbering as in Kabat, refers to the numbering system used for heavy chain variable domains or light chain variable domains of the compilation of antibodies in Kabat et al. (Sequences of Immunological Interest. 5th Ed., 1991, National Institutes of Health, Bethesda, Md.), (“Kabat”). Using this numbering system, the actual linear amino acid sequence can contain fewer or additional amino acids corresponding to a shortening of, or insertion into, a FR or CDR of the variable domain. For example, a heavy chain variable domain can include a single amino acid insert (residue 52a according to Kabat) after residue 52 of H2 and inserted residues (e.g. residues 82a, 82b, and 82c, etc. according to Kabat) after heavy chain FR residue 82. The Kabat numbering of residues can be determined for a given antibody by alignment at regions of homology of the sequence of the antibody with a “standard” Kabat numbered sequence. Chothia refers instead to the location of the structural loops (Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

| Loop | Kabat | AbM | Chothia |
|---|---|---|---|
| L1 | L24-L34 | L24-L34 | L24-L34 |
| L2 | L50-L56 | L50-L56 | L50-L56 |
| L3 | L89-L97 | L89-L97 | L89-L97 |
| H1 | H31-H35B | H26-H35B | H26-H32 . . . 34 |

(Kabat Numbering)

| | | | |
|---|---|---|---|
| H1 | H31-H35 | H26-H35 | H26-H32 |

(Chothia Numbering)

| | | | |
|---|---|---|---|
| H2 | H50-H65 | H50-H58 | H52-H56 |
| H3 | H95-H102 | H95-H102 | H95-H102 |

The term “human” antibody or antigen-binding fragment thereof means an antibody or antigen-binding fragment thereof produced by a human or an antibody or antigen-binding fragment thereof having an amino acid sequence corresponding to an antibody or antigen-binding fragment thereof produced by a human made using any technique known in the art. This definition of a human antibody or antigen-binding fragment thereof includes intact or full-length antibodies and fragments thereof.

“Binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure.

By “specifically binds,” it is generally meant that an antibody binds to an epitope via its antigen binding domain, and that the binding entails some complementarity between the antigen binding domain and the epitope. According to this definition, an antibody is said to “specifically bind” to an epitope when it binds to that epitope, via its antigen binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.”

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this disclosure are based upon antibodies, in some aspects, the polypeptides can occur as single chains or associated chains.

The term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

The term “ideal body weight” (IBW) refers to a size descriptor that is unrelated to total body weight. IBW is an estimate of weight corrected for sex and height, and optionally frame size. IBW can be calculated, for example, using the formulas IBW=0.9H-88 (for males) and IBW=0.9H-92 (for females), wherein H=height in cm.

The term “adjusted ideal body weight” (AIBW) or “adjusted body weight” (ADJ) refers to a size descriptor that accounts for sex, total body weight, and height. AIBW and ADJ are used interchangeably throughout the specification. AIBW (ADJ) can be calculated, for example, using the formula ADJ=IBW+0.4(weight in kg−IBW).

IBW and AIBW (ADJ) are discussed in more detail in Green and Duffull, British Journal of Clinical Pharmacology 58: 119-133 (2004), which is herein incorporated by reference in its entirety.

The term “immunoconjugate” or “conjugate” as used herein refers to a compound or a derivative thereof that is linked to a cell binding agent and is defined by a generic formula: C-L-A, wherein C=cytotoxin, L=linker, and A=antibody or antigen-binding fragment there of (e.g., an anti-FRα antibody or antibody fragment). Immunoconjugates can also be defined by the generic formula in reverse order: A-L-C.

A “linker” is any chemical moiety that is capable of linking a compound, usually a drug, such as maytansinoid, to a cell-binding agent such as an anti-FRα antibody or antigen-binding fragment thereof in a stable, covalent manner. Linkers can be susceptible to or be substantially resistant to cleavage (e.g., acid-induced cleavage, light-induced cleavage, peptidase-induced cleavage, esterase-induced cleavage, or disulfide bond cleavage) at conditions under which the compound or the antibody remains active. Suitable linkers are well known in the art and include, for example, disulfide groups and thioether groups.

As used herein, the term “cytotoxic agent” refers to a substance that inhibits or prevents one or more cellular functions and/or causes cell death. In some aspects, the cytotoxic agent is a maytansinoid, e.g., DM4 or DM1. Immunoconjugates comprising DM4 are disclosed in WO 2011/106528 and WO 2018/160539 A1, each of which is herein incorporated by reference in its entirety.

The terms “cancer” and “cancerous” refer to or describe the physiological condition in mammals in which a population of cells are characterized by unregulated cell growth. Examples of cancer include, but are not limited to, carcinoma, lymphoma, blastoma, sarcoma, and leukemia. The cancer can be a recurrent platinum-sensitive ovarian cancer (rPSOC). In some aspects, platinum-sensitive disease is a disease with progression and/or relapse greater than 6 months from last dose of most recent platinum therapy. In some aspects, platinum-sensitive disease is a disease with progression greater than 6 months from last dose of most recent platinum therapy. In some aspects, platinum-sensitive disease is a disease with radiographic progression greater than 6 months from last dose of most recent platinum therapy. In some aspects, platinum-sensitive disease is a disease with relapse greater than 6 months from last dose of most recent platinum therapy. More particular examples of cancers include fallopian tube cancer, squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancers. The cancer can be a recurrent (relapsed) platinum-sensitive ovarian cancer (rPSOC) such as an epithelial ovarian cancer (EOC) including a high-grade serous EOC. Primary peritoneal cancer and fallopian tube cancer are examples of rPSOCs. The cancer can be a cancer that expresses FRα (“FRα-expressing cancer”).

The terms “cancer cell,” “tumor cell,” and grammatical equivalents refer to the total population of cells derived from a tumor or a pre-cancerous lesion, including both non-tumorigenic cells, which comprise the bulk of the tumor cell population, and tumorigenic stem cells (cancer stem cells). As used herein, the term “tumor cell” will be modified by the term “non-tumorigenic” when referring solely to those tumor cells lacking the capacity to renew and differentiate to distinguish those tumor cells from cancer stem cells.

A “recurrent” cancer is one that has regrown, either at the initial site or at a distant site, after a response to initial therapy.

A “relapsed” patient is one who has signs or symptoms of cancer after remission. Optionally, the patient has relapsed after adjuvant or neoadjuvant therapy.

The term “maintenance therapy” refers to therapy that is given to help keep cancer from coming back after it has disappeared following the initial therapy.

PFS, DFS, and OS can be measured by standards set by the National Cancer Institute and the U.S. Food and Drug Administration for the approval of new drugs. See Johnson et al, (2003) J. Clin. Oncol. 21(7):1404-1411.

“Progression free survival” (PFS) refers to the time from enrollment to disease progression or death. PFS is generally measured using the Kaplan-Meier method and Response Evaluation Criteria in Solid Tumors (RECIST) 1.1 standards. Generally, progression free survival refers to the situation wherein a patient remains alive, without the cancer getting worse.

“Time to Tumor Progression” (TTP) is defined as the time from enrollment to disease progression. TTP is generally measured using the RECIST 1.1 criteria.

A “complete response” or “complete remission” or “CR” indicates the disappearance of all signs of tumor or cancer in response to treatment. This does not always mean the cancer has been cured.

A “partial response” or “PR” refers to a decrease in the size or volume of one or more tumors or lesions, or in the extent of cancer in the body, in response to treatment.

“Stable disease” refers to disease without progression or relapse. In stable disease there is neither sufficient tumor shrinkage to qualify for partial response nor sufficient tumor increase to qualify as progressive disease.

“Progressive disease” refers to the appearance of one more new lesions or tumors and/or the unequivocal progression of existing non-target lesions. Progressive disease can also revert to a tumor growth of more than 20 percent since treatment began, either due to an increases in mass or in spread of the tumor.

“Disease free survival” (DFS) refers to the length of time during and after treatment that the patient remains free of disease.

“Overall Survival” (OS) refers to the time from patient enrollment to death or censored at the date last known alive. OS includes a prolongation in life expectancy as compared to naive or untreated individuals or patients. Overall survival refers to the situation wherein a patient remains alive for a defined period of time, such as one year, five years, etc., e.g., from the time of diagnosis or treatment.

“Clinical benefit” is achievement by a patient of complete response, partial response, or stable disease for a time equal to or longer than 24 weeks.

A “decrease in CA125 levels” can be assessed according to the Gynecologic Cancer Intergroup (GCIG) guidelines. For example, CA125 levels can be measured prior to treatment to establish a baseline CA125 level. CA125 levels can be measured one or more times during or after treatment, and a reduction in the CA125 levels over time as compared to the baseline level is considered a decrease in CA125 levels.

The term “pharmaceutical formulation” refers to a preparation which is in such form as to permit the biological activity of the active ingredient to be effective, and which contains no additional components which are unacceptably toxic to a subject to which the formulation would be administered. The formulation can be sterile.

An “effective amount” of an antibody, immunoconjugate, or other drug as disclosed herein is an amount sufficient to carry out a specifically stated purpose.

The term “therapeutically effective amount” refers to an amount of an antibody, immunoconjugate, or other drug effective to “treat” a disease or disorder in a subject or mammal.

Terms such as “treating” or “treatment” or “to treat” or “alleviating” or “to alleviate” refer to therapeutic measures that cure, slow down, lessen symptoms of, and/or halt progression of a diagnosed pathologic condition or disorder. Thus, those in need of treatment include those already diagnosed with or suspected of having the disorder. In some aspects, a subject is successfully “treated” for cancer according to the methods of the present disclosure if the patient shows one or more of the following: a reduction in the number of or complete absence of cancer cells; a reduction in the tumor size; inhibition of or an absence of cancer cell infiltration into peripheral organs including, for example, the spread of cancer into soft tissue and bone; inhibition of or an absence of tumor metastasis; inhibition or an absence of tumor growth; relief of one or more symptoms associated with the specific cancer; reduced morbidity and mortality; improvement in quality of life; reduction in tumorigenicity, tumorigenic frequency, or tumorigenic capacity, of a tumor; reduction in the number or frequency of cancer stem cells in a tumor; differentiation of tumorigenic cells to a non-tumorigenic state; increased progression-free survival (PFS), disease-free survival (DFS), or overall survival (OS), complete response (CR), partial response (PR), stable disease (SD), a decrease in progressive disease (PD), a reduced time to progression (TTP), or any combination thereof. In some aspects, a subject (e.g., a human patient) in need thereof is successfully treated according to the methods of the present disclosure if the subject is cured of, shows relief of, has reduced symptoms of, and/or shows no progression of a folate receptor alpha (FRα)-expressing recurrent platinum-sensitive ovarian cancer (rPSOC). In certain aspects, a subject in need thereof is successfully “treated” for rPSOC according to the methods of the present disclosure if the method increases the subject's overall survival. In certain aspects, a subject in need thereof is successfully “treated” for rPSOC according to the methods of the present disclosure if the method results in a decrease in tumor size. In certain aspects, a subject in need thereof is successfully “treated” for rPSOC according to the methods of the present disclosure if the method results in a decrease in cancer antigen 125 (CA-125) expression. In certain aspects, a subject in need thereof is successfully “treated” for rPSOC according to the methods of the present disclosure if the method results in the subject achieving a duration of response (DOR) of at least 4 months. In certain aspects, a subject in need thereof is successfully “treated” for rPSOC according to the methods of the present disclosure if the method results in the subject achieving a progression-free survival (PFS) of at least 3.98 months. In certain aspects, a subject in need thereof is successfully “treated” for rPSOC according to the methods of the present disclosure if the method results in an about 80% chance of the subject achieving a progression-free survival (PFS) of at least 3 months. In certain aspects, a subject in need thereof is successfully “treated” for rPSOC according to the methods of the present disclosure if the method results in an about 61% chance of the subject achieving a progression-free survival (PFS) of at least 6 months. In certain aspects, a subject in need thereof is successfully “treated” for rPSOC according to the methods of the present disclosure if the method results in an about 39% chance of the subject achieving a progression-free survival (PFS) of at least 9 months. In certain aspects, a subject in need thereof is successfully “treated” for rPSOC according to the methods of the present disclosure if the method results in an about 19% chance of the subject achieving a progression-free survival (PFS) of at least 12 months. In certain aspects, a subject in need thereof is successfully “treated” for rPSOC according to the methods of the present disclosure if the subject is administered the treatment that has been shown to cure, slow down, lessen symptoms of, and/or halt progression of rPSOC. In certain aspects, a subject in need thereof is successfully “treated” for rPSOC according to the methods of the present disclosure if the subject is administered the treatment that has been shown to result in a CA125 response rate between about 60% and about 86%. In certain aspects, a subject in need thereof is successfully “treated” for rPSOC according to the methods of the present disclosure if the subject is administered the treatment that has been shown to result in a CA125 response rate of about 75%. In certain aspects, a subject in need thereof is successfully “treated” for rPSOC according to the methods of the present disclosure if the subject is administered the treatment that has been shown to result in an objective response rate (ORR) between 40% and 65%. In certain aspects, a subject in need thereof is successfully “treated” for rPSOC according to the methods of the present disclosure if the subject is administered the treatment that has been shown to result in an objective response rate (ORR) of about 52%. In certain aspects, a subject in need thereof is successfully “treated” for rPSOC according to the methods of the present disclosure if the subject is administered the treatment that has been shown to result in an duration of response (DOR) of 4.63 months. In certain aspects, a subject in need thereof is successfully “treated” for rPSOC according to the methods of the present disclosure if the subject is administered the treatment that has been shown to result in an duration of response (DOR) of 8.25 months. In certain aspects, a subject in need thereof is successfully “treated” for rPSOC according to the methods of the present disclosure if the subject is administered the treatment that has been shown to result in a progression-free survival (PFS) of 3.98 months. In certain aspects, a subject in need thereof is successfully “treated” for rPSOC according to the methods of the present disclosure if the subject is administered the treatment that has been shown to result in a progression-free survival (PFS) of about 6.93 months. In certain aspects, rPSOC is epithelial ovarian cancer (EOC). In certain aspects, rPSOC is high-grade serous EOC. In certain aspects, rPSOC is primary peritoneal cancer. In certain aspects, rPSOC is fallopian tube cancer.

The terms “administer,” “administering,” “administration,” and the like, as used herein, refer to methods that may be used to enable delivery of the immunoconjugate to the desired site of biological action. Administration techniques that can be employed with the agents and methods described herein are found in e.g., Goodman and Gilman, The Pharmacological Basis of Therapeutics, current ed.; Pergamon; and Remington's, Pharmaceutical Sciences (current edition), Mack Publishing Co., Easton, Pa. In one aspect, immunoconjugate is administered intravenously.

As used herein, the terms “about” and “approximately,” when used to modify a numeric value or numeric range, indicate that deviations of up to 10% above and down to 10% below the value or range remain within the intended meaning of the recited value or range. It is understood that wherever aspects are described herein with the language “about” or “approximately” a numeric value or range, otherwise analogous aspects referring to the specific numeric value or range (without “about”) are also provided.

The recitation of a listing of chemical groups in any definition of a variable herein includes definitions of that variable as any single group or combination of listed groups. The recitation of an aspect herein includes that aspect as any single aspect or in combination with any other aspects or portions thereof.

As used in the present disclosure and claims, the singular forms “a,” “an,” and “the” include plural forms unless the context clearly dictates otherwise.

It is understood that wherever aspects are described herein with the language “comprising,” otherwise analogous aspects described in terms of “consisting of” and/or “consisting essentially of” are also provided. In this disclosure, “comprises,” “comprising,” “containing” and “having” and the like can mean “includes,” “including,” and the like; “consisting essentially of” or “consists essentially” are open-ended, allowing for the presence of more than that which is recited so long as basic or novel characteristics of that which is recited is not changed by the presence of more than that which is recited, but excludes prior art aspects.

Unless specifically stated or obvious from context, as used herein, the term “or” is understood to be inclusive. The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following: A, B, and C; A, B, or C; A or C; A or B; B or C; A and C; A and B; B and C; A (alone); B (alone); and C (alone).

Any compositions or methods provided herein can be combined with one or more of any of the other compositions and methods provided herein.

### II. Anti-FRα Antibodies and Antigen-Binding Fragments Thereof

As described herein, anti-FRα antibodies and antigen-binding fragments thereof can be used therapeutically (e.g., to treat cancer) and/or to detect FRα (e.g., membrane FRα). Exemplary anti-FRα antibodies and antigen binding fragments thereof are known in the art and have been disclosed, for example, in WO 2011/106528, WO 2014/036495, WO 2015/031815, and U.S. Published Application No. 2020/0362029, each of which is herein incorporated by reference in its entirety. Additional anti-FRα antibodies and antigen binding fragments thereof are known in the art and have been disclosed, for example, in WO 2012/061759, U.S. Published Application No. 2019/0233512, U.S. Published Application No. 2020/0147229, U.S. Published Application No. 2020/0297860 U.S. Published Application No. 2020/0353076, U.S. Pat. Nos. 10,822,410 and 10,101,343, each of which is herein incorporated by reference in its entirety.

Described herein are methods of administering immunoconjugates that specifically bind FRα (e.g., IMGN853 and mirvetuximab soravtansine). These agents are referred to herein as “FOLR1-immunoconjugates or anti-FOLR1 immunoconjugates” and “FRα immunoconjugates or anti-FRα immunoconjugates”

Anti-FRα immunoconjugates contain a cell binding agent linked to a cytotoxin. The cell binding agents can be anti-FRα antibodies or antigen-binding fragments thereof. Examples of therapeutically effective anti-FOLR1 antibodies can be found in US Appl. Pub. No. US 2012/0009181 which is herein incorporated by reference. An example of a therapeutically effective anti-FOLR1 antibody is huMov19 (M9346A) (comprising the sequences of SEQ ID NO: 19 and SEQ ID NO:20). In certain aspects, the huMov19 anti-FRα antibody is comprised of a variable domain heavy chain represented by SEQ ID NO:19 and a variable domain light chain represented by SEQ ID NO:20 (version 1.60 of huMov19). In certain aspects, the huMov19 (M9346A) antibody is encoded by the plasmids deposited with the American Type Culture Collection (ATCC), located at 10801 University Boulevard, Manassas, VA 20110 on Apr. 7, 2010 under the terms of the Budapest Treaty and having ATCC deposit nos. PTA-10772 and PTA-10773 or 10774.

Immunodetection assay kits for the immunodetection of FRα expression are commercially available. See, e.g., the Ventana FOLR1 (FOLR1-2.1) CDx assay. In some aspects, expression of FRα is measured by IHC and performed using the FOLR1-2.1 detection antibody. The FOLR1-2.1 detection antibody is commercially available as part of the Ventana FOLR1 (FOLR1-2.1) CDx assay.

Amino acid sequences of huMov19 and 2.1 are provided in Tables 1-6 below.

TABLE 1
Variable heavy chain CDR amino acid sequences

| Antibody | VH-CDR1 | VH-CDR2 | VH-CDR3 |
|---|---|---|---|
| huMov19 | Kabat Defined: | Kabat Defined: | Kabat Defined: |
| (M9346A) | GYFMN (SEQ ID | RIHPYDGDTFYNQKFQG | YDGSRAMDY (SEQ ID |
| | NO: 3) | (SEQ ID NO: 4) | NO: 5) |
| | AbM Defined: | AbM Defined: | AbM Defined: |
| | GYTFTGYFMN | RIHPYDGDTF | YDGSRAMDY |
| | (SEQ ID NO: 9) | (SEQ ID NO: 10) | (SEQ ID NO: 11) |
| muMOV19 | | Kabat Defined | |
| | | RIHPYDGDTFYNQNFKD | |
| | | (SEQ ID NO: 12) | |
| muFRIHC2- | NSYIH | WIYPESLNTQYNEKFKA | RGIYYYSPYALDH |
| 1 (″2.1″) | (SEQ ID NO: 13) | (SEQ ID NO: 14) | (SEQ ID NO: 15) |

TABLE 2
Variable light chain CDR amino acid sequences

| Antibody | VL-CDR1 | VL-CDR2 | VL-CDR3 |
|---|---|---|---|
| huMov19 | KASQSVSFAGTSLMH | RASNLEA | QQSREYPYT |
| (M9346A) | (SEQ ID NO: 6) | (SEQ ID NO: 7) | (SEQ ID NO: 8) |
| muFRIHC2- | KSSKSLLNSDGFTYLD | LVSNHFS (SEQ ID | FQSNYLPLT (SEQ ID |
| 1 (″2.1″) | (SEQ ID NO: 16) | NO: 17) | NO: 18) |

TABLE 3
Variable heavy chain (VH) amino acid sequences

| Antibody | Amino Acid Sequence |
|---|---|
| huMov19 | QVQLVQSGAEVVKPGASVKISCKASGYTFTGYFMNWVKQSPGQSLE |
| | WIGRIHPYDGDTFYNQKFQGKATLTVDKSSNTAHMELLSLTSEDFAV |
| | YYCTRYDGSRAMDYWGQGTTVTVSS (SEQ ID NO: 19) |
| muFRIHC2-1 | QVQLQQSGPELVKPGASVRISCKASGYTFTNSYIHWVKKRPGQGLEW |
| (″2.1″) | IGWIYPESLNTQYNEKFKAKATLTADKSSSTSYMQLSSLTSEDSAVYF |
| | CARRGIYYYSPYALDHWGQGASVTVSS (SEQ ID NO: 21) |

TABLE 4
Variable light chain (VL) amino acid sequences

| Antibody | Amino Acid Sequence |
|---|---|
| huMov19 | DIVLTQSPLSLAVSLGQPAIISCKASQSVSFAGTSLMHWYHQKPGQQP |
| | RLLIYRASNLEAGVPDRFSGSGSKTDFTLTISPVEAEDAATYYCQQSR |
| | EYPYTFGGGTKLEIKR (SEQ ID NO: 20) |
| muFRIHC2-1 | SDVVLTQTPLSLPVNIGDQASISCKSSKSLLNSDGFTYLDWYLQKPGQ |
| (″2.1″) | SPQLLIYLVSNHFSGVPDRFSGSGSGTDFTLKISRVEAEDLGVYYCFQS |
| | NYLPLTFGGGTKLEIKR (SEQ ID NO: 22) |

TABLE 5
Full-length heavy chain amino acid sequences

| Antibody | Full-Length Amino Acid Sequence |
|---|---|
| huMov19 | QVQLVQSGAEVVKPGASVKISCKASGYTFTGYFMNWVKQSPGQSLE |
| | WIGRIHPYDGDTFYNQKFQGKATLTVDKSSNTAHMELLSLTSEDFAV |
| | YYCTRYDGSRAMDYWGQGTTVTVSSASTKGPSVFPLAPSSKSTSGGT |
| | AALGCLVKDYFPEPVTVSWNSGALTSGVHTFPAVLQSSGLYSLSSVV |
| | TVPSSSLGTQTYICNVNHKPSNTKVDKKVEPKSCDKTHTCPPCPAPEL |
| | LGGPSVFLFPPKPKDTLMISRTPEVTCVVVDVSHEDPEVKFNWYVDG |
| | VEVHNAKTKPREEQYNSTYRVVSVLTVLHQDWLNGKEYKCKVSNK |
| | ALPAPIEKTISKAKGQPREPQVYTLPPSRDELTKNQVSLTCLVKGFYPS |
| | DIAVEWESNGQPENNYKTTPPVLDSDGSFFLYSKLTVDKSRWQQGN |
| | VFSCSVMHEALHNHYTQKSLSLSPG (SEQ ID NO: 23) |
| muFRIHC2-1 | QVQLQQSGPELVKPGASVRISCKASGYTFTNSYIHWVKKRPGQGLEW |
| (″2.1″) | IGWIYPESLNTQYNEKFKAKATLTADKSSSTSYMQLSSLTSEDSAVYF |
| | CARRGIYYYSPYALDHWGQGASVTVSSAKTTPPSVYPLAPGSAAQTN |
| | SMVTLGCLVKGYFPEPVTVTWNSGSLSSGVHTFPAVLESDLYTLSSS |
| | VTVPSSMRPSETVTCNVAHPASSTKVDKKIVPRDCGCKPCICTVPEVS |
| | SVFIFPPKPKDVLTITLTPKVTCVVVDISKDDPEVQFSWFVDDVEVHT |
| | AQTQPREEQFNSTFRSVSELPIMHQDWLNGKEFKCRVNSAAFPAPIEK |
| | TISKTKGRPKAPQVYTIPPPKEQMAKDKVSLTCMITDFFPEDITVEWQ |
| | WNGQPAENYKNTQPIMNTNGSYFVYSKLNVQKSNWEAGNTFTCSVL |
| | HEGLHNHHTEKSLSHSPGK (SEQ ID NO: 24) |

TABLE 6
Full-length light chain amino acid sequences

| Antibody | Full-Length Amino Acid Sequence |
|---|---|
| huMov19 | DIVLTQSPLSLAVSLGQPAIISCKASQSVSFAGTSLMHWYHQKPGQQP |
| | RLLIYRASNLEAGVPDRFSGSGSKTDFTLTISPVEAEDAATYYCQQSR |
| | EYPYTFGGGTKLEIKRTVAAPSVFIFPPSDEQLKSGTASVVCLLNNFYP |
| | REAKVQWKVDNALQSGNSQESVTEQDSKDSTYSLSSTLTLSKADYE |
| | KHKVYACEVTHQGLSSPVTKSFNRGEC (SEQ ID NO: 25) |
| muFRIHC2-1 | SDVVLTQTPLSLPVNIGDQASISCKSSKSLLNSDGFTYLDWYLQKPGQ |
| (″2.1″) | SPQLLIYLVSNHFSGVPDRFSGSGSGTDFTLKISRVEAEDLGVYYCFQS |
| | NYLPLTFGGGTKLEIKRADAAPTVSIFPPSSEQLTSGGASVVCFLNNFY |
| | PKDINVKWKIDGSERQNGVLNSWTDQDSKDSTYSMSSTLTLTKDEYE |
| | RHNSYTCEATHKTSTSPIVKSFNRNEC (SEQ ID NO: 26) |

In some aspects, the anti-FRα immunoconjugates disclosed herein comprise humanized antibodies or antigen-binding fragments thereof. In some aspects, the humanized antibody or fragment is a resurfaced antibody or antigen-binding fragment thereof. In other aspects, the anti-FRα immunoconjugates comprises a fully human antibody or antigen-binding fragment thereof.

In certain aspects, the anti-FRα immunoconjugates have one or more of the following effects: inhibit proliferation of tumor cells, reduce the tumorigenicity of a tumor by reducing the frequency of cancer stem cells in the tumor, inhibit tumor growth, increase patient survival, trigger cell death of tumor cells, differentiate tumorigenic cells to a non-tumorigenic state, or prevent or reduce metastasis of tumor cells.

In certain aspects, the anti-FRα immunoconjugate comprises an antibody that has antibody-dependent cellular cytotoxicity (ADCC) activity.

In some aspects, the anti-FRα immunoconjugates are capable of reducing tumor volume. The ability of an anti-FRα immunoconjugate to reduce tumor volume can be assessed, for example, by measuring a % T/C value, which is the median tumor volume of treated subjects divided by the median tumor volume of the control subjects. In certain aspects, immunoconjugates or other agents that specifically bind human FRα trigger cell death via a cytotoxic agent. For example, in certain aspects, an antibody to a human FRαantibody is conjugated to a maytansinoid that is activated in tumor cells expressing the FRα by protein internalization. In certain aspects, the anti-FRα immunoconjugates are capable of inhibiting tumor growth. In certain aspects, the anti-FRα immunoconjugates are capable of inhibiting tumor growth in vivo (e.g., in a xenograft mouse model and/or in a human having cancer). In certain aspects, the anti-FRαimmunoconjugates are capable of decreasing CA125 in ovarian cancer patients.

In some aspects, the anti-FRα immunoconjugates disclosed herein comprise an antibody or an antigen-binding fragment thereof that specifically binds a human FRα. In some aspects, the antibody or an antigen-binding fragment thereof that specifically binds a human FRα can comprise any of the variable heavy chain CDR amino acid sequences and/or any of the variable light chain CDR amino acid sequences described herein, in any combination. In some aspects, the anti-FRα antibody or antigen-binding fragment thereof comprises the sequences of SEQ ID NOs:3-8. In some aspects, the anti-FRα antibody or antigen-binding fragment thereof comprises the sequences of SEQ ID NOs:9-11 and 6-8. In some aspects, the anti-FRα antibody or antigen-binding fragment thereof comprises any combination of the sequences of SEQ ID NOs:3-5, 6-8, 9-11, and/or 12. In some aspects, the antibody or an antigen-binding fragment thereof that specifically binds a human FRα can comprise any of the Kabat-defined variable heavy chain CDR amino acid sequences, the Abm-defined variable heavy chain CDR amino acid sequences and/or any of the Kabat-defined variable light chain CDR amino acid sequences, or any of the Abm-defined variable light chain CDR amino acid sequences described herein, in any combination.

In some aspects, the anti-FRα antibody or antigen-binding fragment thereof can comprise one of the individual light chains or heavy chains described herein. Anti-FRα antibodies also comprise both a light chain and a heavy chain. In some aspects, the antibody or an antigen-binding fragment thereof that specifically binds a human FRα comprises a VH comprising the sequence of SEQ ID NO:19 and a VL comprising the sequence of SEQ ID NO:20. In some aspects, the antibody or an antigen-binding fragment thereof that specifically binds a human FRα comprises a full length heavy chain comprising SEQ ID NO:23, and a full length light chain comprising SEQ ID NO:24.

In some aspects, an anti-FRα-antibody or antigen-binding fragment thereof comprises a heavy chain constant region, such as an IgG1, IgG2, IgG3, IgG4, IgA, IgE, IgM or IgD constant region. In some aspects, the heavy chain constant region is an IgG1 heavy chain constant region. Furthermore, in some aspects, an anti-FRα-antibody or antigen-binding fragment thereof can comprise a light chain constant region, either a kappa light chain constant region or a lambda light chain constant region. In some aspects, the light chain constant region is a kappa light chain constant region. In some aspects, the antibody or an antigen-binding fragment thereof that specifically binds a human FRα comprises an IgG1 antibody comprising a full length heavy chain comprising SEQ ID NO:23, and a full length light chain comprising SEQ ID NO:24.

In some aspects, an anti-FRα immunoconjugate can comprise a maytansinoid linked to an anti-FRα antibody or antigen-binding fragment thereof comprising (a) a heavy chain (HC) comprising the same amino acid sequence as the amino acid sequence of the heavy chain encoded by the plasmid deposited with the American Type Culture Collection (ATCC) as PTA-10772 and (b) a light chain (LC) comprising the same amino acid sequence as the amino acid sequence of the light chain encoded by the plasmid deposited with the ATCC as PTA-10774, wherein the maytansinoid is DM4 and/or the maytansinoid is linked to the antibody or antigen-binding fragment thereof via a sulfo-SPDB linker.

This invention also encompasses bispecific antibodies that specifically recognize a FRα. Bispecific antibodies are antibodies that are capable of specifically recognizing and binding at least two different epitopes. The different epitopes can either be within the same molecule (e.g. the same FRα) or on different molecules such that both, for example, the antibodies can specifically recognize and bind a FRA as well as, for example, 1) an effector molecule on a leukocyte such as a T-cell receptor (e.g. CD3) or Fc receptor (e.g. CD64, CD32, or CD16) or 2) a cytotoxic agent as described in detail below.

The polypeptides of the present invention can be recombinant polypeptides, natural polypeptides, or synthetic polypeptides comprising an antibody, or fragment thereof, against a human FRα.

The polypeptides and analogs can be further modified to contain additional chemical moieties not normally part of the protein. Those derivatized moieties can improve the solubility, the biological half-life or absorption of the protein. The moieties can also reduce or eliminate any desirable side effects of the proteins and the like. An overview for those moieties can be found in REMINGTON'S PHARMACEUTICAL SCIENCES, 20th ed., Mack Publishing Co., Easton, PA (2000).

Methods known in the art for purifying antibodies and other proteins also include, for example, those described in U.S. Patent Publication No. 2008/0312425, 2008/0177048, and 2009/0187005, each of which is hereby incorporated by reference herein in its entirety.

A. Methods for Treating FRα-Expressing rPSOCs

The anti-FRα antibodies and immunoconjugates disclosed herein are useful in a variety of applications including, but not limited to, therapeutic treatment methods, such as the treatment of cancer. In certain aspects, the agents are useful for inhibiting tumor growth, inducing differentiation, inhibiting metastases, reducing tumor volume, and/or reducing the tumorigenicity of a tumor. The methods of use can be in vivo methods.

In some aspects, the present disclosure provides methods for treating a human patient having a folate receptor alpha (FRα)-expressing recurrent (relapsed) platinum-sensitive ovarian cancer (rPSOC). In some aspects, the patient has had at least two lines of therapy prior to initiation of the present method. In some aspects, the patient has had at least three lines of therapy prior to initiation of the present method. In some aspects, the patient has had at least four lines of therapy prior to initiation of the present method. In some aspects, rPSOC is ovarian cancer with progression greater than 6 months from last dose of most recent platinum therapy. In some aspects, rPSOC is ovarian cancer with radiographic progression greater than 6 months from last dose of most recent platinum therapy.

In some aspects, the patient has had Poly (ADP-ribose) polymerase (PARP) inhibitor (PARPi) therapy prior to initiation of the present method. In some aspects, the PARPi comprises olaparib, talazoparib, rucaparib, niraparib, or a combination thereof. In some aspects, the PARPi comprises olaparib. In some aspects, the PARPi comprises olaparib in combination with another PARPi. In some aspects, the PARPi comprises talazoparib. In some aspects, the PARPi comprises talazoparib in combination with another PARPi. In some aspects, the PARPi comprises rucaparib. In some aspects, the PARPi comprises rucaparib in combination with another PARPi. In some aspects, the PARPi comprises niraparib. In some aspects, the PARPi comprises niraparib in combination with another PARPi. In some aspects, the patient has had disease progression (PD) while receiving PARPi therapy or within 30 days of receiving PARPi prior to initiation of the present method. In some aspects, the patient has had no disease progression (PD) while receiving PARPi therapy or within 30 days of receiving PARPi prior to initiation of the present method. In some aspects, the patient has had disease recurrence while receiving PARPi therapy or within 30 days of receiving PARPi prior to initiation of the present method. In some aspects, the patient has had no disease recurrence while receiving PARPi therapy or within 30 days of receiving PARPi prior to initiation of the present method.

In some aspects, the patient has had bevacizumab therapy prior to initiation of the present method. In some aspects, the patient has had at least one line of therapy comprising bevacizumab prior to initiation of the present method. In some aspects, the patient has had more than one line of therapy comprising bevacizumab prior to initiation of the present method. In some aspects, the patient has had at least one line of VEGF antagonist therapy comprising a VEGF pathway antagonist agent (e.g., bevacizumab, ramucirumab, ziv-aflibercept, regorafenib, sunitinib, sorafenib, brivanib, and cabozantnib) prior to initiation of the present method. In some aspects, the patient has had more than one line of therapy comprising a VEGF pathway antagonist agent prior to initiation of the present method.

In some aspects, the patient has had taxane therapy prior to initiation of the present method. In some aspects, the taxane therapy comprises paclitaxel, docetaxel, cabazitaxel, or a combination thereof. In some aspects, taxane therapy comprises paclitaxel. In some aspects, the taxane therapy comprises paclitaxel and another taxane. In some aspects, the taxane therapy comprises docetaxel. In some aspects, the taxane therapy comprises docetaxel and another taxane. In some aspects, the taxane therapy comprises cabazitaxel. In some aspects, the taxane therapy comprises cabazitaxel and another taxane. In some aspects, the patient has had at least one line of therapy comprising taxane prior to initiation of the present method. In some aspects, the patient has had more than one line of therapy comprising taxane prior to initiation of the present method.

In some aspects, methods for treating a human patient having a folate receptor alpha (FRα)-expressing recurrent (relapsed) platinum-sensitive ovarian cancer (rPSOC) comprise administering to the patient an effective amount of an immunoconjugate having a formula (A)-(L)-(C), wherein: (A) is an antibody or an antigen-binding fragment thereof that specifically binds a human FRα, comprising a heavy chain variable region (VH) complementary determining region (CDR)1 sequence of SEQ ID NO: 3, a VH CDR2 sequence of SEQ ID NO: 4, and a VH CDR3 sequence of SEQ ID NO: 5, a light chain variable region (VL) CDR1 sequence of SEQ ID NO: 6, a VL CDR2 sequence of SEQ ID NO: 7, and a VL CDR3 sequence of SEQ ID NO: 8; (L) is the linker N-succinimidyl 4-(2-pyridyldithio)-2-sulfobutanoate (sulfo-SPDB); and (C) is a maytansinoid; wherein L links A to C, and wherein the patient has had least two lines of therapy prior to administering the immunoconjugate.

In some aspects, the rPSOC is epithelial ovarian cancer (EOC). In some aspects, the rPSOC is high-grade serous EOC. In some aspects, the rPSOC is primary peritoneal cancer. In some aspects, the rPSOC is fallopian tube cancer.

In some aspects, the patient has had at least one line of platinum-containing therapy prior to administering the immunoconjugate. In some aspects, the patient has had one independent non-platinum cytotoxic therapy prior to administering the immunoconjugate. In some aspects, the patient has a platinum allergy. In some aspects, the patient has had at least two lines of platinum-containing therapy prior to administering the immunoconjugate. In some aspects, the patient has had a platinum-free interval of at least 6 months prior to initiation of the present method. In some aspects, the patient has had a platinum-free interval of between 6 months and 12 months prior to initiation of the present method. In some aspects, the patient has had a platinum-free interval of at least 6 months prior to initiation of the present method. In some aspects, the patient has had a platinum-free interval of between 6 months and 12 months prior to initiation of the present method. In some aspects, the patient has had a platinum-free interval of more than 12 months prior to initiation of the present method. In some aspects, platinum-free interval is the time from last dose of the latest line platinum therapy to the date of disease progression and/or relapse following that line of therapy (time rounded to whole number). In some aspects, the time from last dose of the first platinum therapy to the date of disease progression following that line of therapy for the patient prior to initiation of the present method is at least 6 months. In some aspects, the time from last dose of the first platinum therapy to the date of disease progression following that line of therapy for the patient is between 6 months and 12 months. In some aspects, the time from last dose of the first platinum therapy to the date of disease recurrence following that line of therapy for the patient is more than 12 months. In some aspects, the time from last dose of the first platinum therapy to the date of disease recurrence following that line of therapy for the patient is between 6 months and 12 months. In some aspects, the time from last dose of the first platinum therapy to the date of disease recurrence following that line of therapy for the patient is more than 12 months.

In some aspects of the methods disclosed herein, the immunoconjugate comprising an anti-FRα antibody or antigen-binding fragment thereof can be administered at particular dosages. For example, the immunoconjugate (e.g., IMGN853) can be administered at a dose of about 4.0 mg/kg to about 6.0 mg/kg, wherein the kilograms of body weight are adjusted ideal body weight (AIBW or ADJ). In some aspects, the immunoconjugate (e.g., IMGN853) can be administered at about 4.2 mg/kg, wherein the kilograms of body weight are adjusted to IBW, LBW, or AIBW (ADJ). In some aspects, the immunoconjugate (e.g., IMGN853) are administered at about 4.5 mg/kg, wherein the kilograms of body weight are adjusted to AIBW (ADJ). In some aspects, the immunoconjugate (e.g., IMGN853) can be administered at about 4.8 mg/kg, wherein the kilograms of body weight are adjusted to AIBW (ADJ). In some aspects, the immunoconjugate (e.g., IMGN853) can be administered at about 5.0 mg/kg, wherein the kilograms of body weight are adjusted to AIBW (ADJ). In some aspects, the immunoconjugate (e.g., IMGN853) can be administered at about 5.5 mg/kg, wherein the kilograms of body weight are adjusted to AIBW (ADJ In some aspects, the immunoconjugate (e.g., IMGN853) can be administered at about 5.6 mg/kg, wherein the kilograms of body weight are adjusted to AIBW (ADJ). In some aspects, the immunoconjugate (e.g., IMGN853) can be administered at about 6.0 mg/kg, wherein the kilograms of body weight are adjusted to AIBW (ADJ).

In particular aspects, the immunoconjugate (e.g., IMGN853) can be administered about once every three weeks.

The immunoconjugate (e.g., IMGN853) can also be administered in an about 3-week (i.e. about 21-day) cycle. For example, the immunoconjugate (e.g., IMGN853) can be administered once in about 3 weeks. Thus, in some aspects, the immunoconjugate (e.g., IMGN853) can be administered at about day 1 of a 21-day cycle. In a particular aspects, the immunoconjugate (e.g., IMGN853) can be administered about once every three weeks.

According to some aspects of the methods disclosed herein, the immunoconjugate (e.g., IMGN853) is administered at a dose of about 4.0 to about 6.0 milligrams (mg) per kilogram (kg) of adjusted ideal body weight (AIBW) of the patient, once every three weeks, on day one of every three-week cycle. In some aspects, the immunoconjugate (e.g., IMGN853) is administered at a dose of about 6.0 milligrams (mg) per kilogram (kg) of adjusted ideal body weight (AIBW) of the patient, once every three weeks, on day one of every three-week cycle. In some aspects, the immunoconjugate (e.g., IMGN853) is administered at a dose of about 5.0 milligrams (mg) per kilogram (kg) of adjusted ideal body weight (AIBW) of the patient, once every three weeks, on day one of every three-week cycle. In some aspects, the immunoconjugate (e.g., IMGN853) is administered at a dose of about 4.0 milligrams (mg) per kilogram (kg) of adjusted ideal body weight (AIBW) of the patient, once every three weeks, on day one of every three-week cycle.

In some aspects, the present disclosure provides a method for treating a human patient having a folate receptor alpha (FRα)-expressing recurrent (relapsed) platinum-sensitive, high-grade serous epithelial ovarian cancer (EOC), comprising administering to the patient an immunoconjugate comprising an antibody or antigen-binding fragment thereof that specifically binds to human FRα, comprising a VH comprising the sequence of SEQ ID NO:19 and a VL comprising the sequence of SEQ ID NO:20, and the maytansinoid DM4, linked via a N-succinimidyl 4-(2-pyridyldithio)-2-sulfobutanoate (sulfo-SPDB) linker, wherein the immunoconjugate is administered at a dose of 6.0 mg/kg AIBW, wherein AIBW is calculated according to the formula: AIBW=ideal body weight (IBW)+0.4 (actual weight in kg−IBW), wherein IBW (male)=0.9H-88 and IBW (female)=0.9H−92, wherein H=height in cm, and wherein the immunoconjugate is administered once every three weeks.

In some aspects, the present disclosure provides a method for treating a human patient having a folate receptor alpha (FRα)-expressing peritoneal cancer, comprising: administering to the patient an immunoconjugate comprising an antibody or antigen-binding fragment thereof that specifically binds to human FRα, comprising a VH comprising the sequence of SEQ ID NO:19 and a VL comprising the sequence of SEQ ID NO:20, and the maytansinoid DM4, linked via a N-succinimidyl 4-(2-pyridyldithio)-2-sulfobutanoate (sulfo-SPDB) linker, wherein the immunoconjugate is administered at a dose of 6.0 mg/kg AIBW, wherein AIBW is calculated according to the formula: AIBW=ideal body weight (IBW)+0.4 (actual weight in kg−IBW), wherein IBW (male)=0.9H-88 and IBW (female)=0.9H− 92, wherein H=height in cm, and wherein the immunoconjugate is administered once every three weeks.

In some aspects, the present disclosure provides a method for treating a human patient having a folate receptor alpha (FRα)-expressing fallopian tube cancer, comprising: administering to the patient an immunoconjugate comprising an antibody or antigen-binding fragment thereof that specifically binds to human FRα, comprising a VH comprising the sequence of SEQ ID NO:19 and a VL comprising the sequence of SEQ ID NO:20, and the maytansinoid DM4, linked via a N-succinimidyl 4-(2-pyridyldithio)-2-sulfobutanoate (sulfo-SPDB) linker, wherein the immunoconjugate is administered at a dose of 6.0 mg/kg AIBW, wherein AIBW is calculated according to the formula: AIBW=ideal body weight (IBW)+0.4 (actual weight in kg−IBW), wherein IBW (male)=0.9H-88 and IBW (female)=0.9H− 92, wherein H=height in cm, and wherein the immunoconjugate is administered once every three weeks.

In some aspects, the administration of the immunoconjugate increases the patient's overall survival.

In some aspects, the administration is a third-line therapy. In some aspects, the administration is a fourth-line therapy.

The present disclosure further provides pharmaceutical compositions comprising one or more of the FRα-immunoconjugates described herein. In certain aspects, the pharmaceutical compositions further comprise a pharmaceutically acceptable vehicle. These pharmaceutical compositions can be used for inhibiting tumor growth and treating cancer in human patients.

In certain aspects, formulations are prepared for storage and use by combining a purified antibody or agent of the present invention with a pharmaceutically acceptable vehicle (e.g. carrier, excipient) (Remington, The Science and Practice of Pharmacy 20th Edition Mack Publishing, 2000). Suitable pharmaceutically acceptable vehicles include, but are not limited to, nontoxic buffers such as phosphate, citrate, and other organic acids; salts such as sodium chloride; antioxidants including ascorbic acid and methionine; preservatives (e.g. octadecyldimethylbenzyl ammonium chloride; hexamethonium chloride; benzalkonium chloride; benzethonium chloride; phenol, butyl or benzyl alcohol; alkyl parabens, such as methyl or propyl paraben; catechol; resorcinol; cyclohexanol; 3-pentanol; and m-cresol); low molecular weight polypeptides (e.g. less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose or sorbitol; salt-forming counter-ions such as sodium; metal complexes (e.g. Zn-protein complexes); and non-ionic surfactants such as TWEEN or polyethylene glycol (PEG).

The pharmaceutical compositions described herein can be administered in any number of ways for systemic treatment. Administration can be intravenous, intraarterial, subcutaneous, intraperitoneal or intramuscular injection or infusion. In some particular aspects, the administration is intravenous. In particular aspects, the administration is via intravenous infusion.

In some aspects of the methods disclosed herein, the immunoconjugate and/or pharmaceutical composition comprising the immunoconjugate can be administered at a rate of between about 0.5 mg/min and about 6 mg/min. In some aspects, the immunoconjugate and/or pharmaceutical composition comprising the immunoconjugate can be administered at a rate of about 1 mg/min. In some aspects, the immunoconjugate and/or pharmaceutical composition comprising the immunoconjugate can be administered at a rate of about 2 mg/min. In some aspects, the immunoconjugate and/or pharmaceutical composition comprising the immunoconjugate can be administered at a rate of about 3 mg/min. In some aspects, the immunoconjugate and/or pharmaceutical composition comprising the immunoconjugate can be administered at a rate of about 4 mg/min. In some aspects, the immunoconjugate and/or pharmaceutical composition comprising the immunoconjugate can be administered at a rate of about 5 mg/min. In some aspects, the immunoconjugate and/or pharmaceutical composition comprising the immunoconjugate can be administered at a rate of about 6 mg/min.

Nonclinical studies have revealed a positive correlation between the level of FRα expression on the cell surface, the amount of maytansinoid catabolites generated, and the degree of sensitivity of the cells to the immunoconjugates described herein (e.g., IMGN853) in vitro. The immunoconjugates described herein (e.g., IMGN853), are not active against low and negative FRα expressing cells. Some patients with rPSOC, primary peritoneal, or fallopian tube cancer, have tumors that express above average, increased, or high levels of FRα. Accordingly, in some aspects, a high level of FRα refers to a level that is above average for patients with rPSOC, primary peritoneal, or fallopian tube cancer.

In some aspects, the FRα protein expression is measured by immunohistochemistry (IHC) and given a staining intensity score and/or a staining uniformity score by comparison to controls (e.g., calibrated controls) exhibiting defined scores (e.g. an intensity score of 3 is given to the test sample if the intensity is comparable to the level 3 calibrated control or an intensity of 2 (moderate) is given to the test sample if the intensity is comparable to the level 2 calibrated control). A staining uniformity that is “heterogeneous” (i.e., at least 25% and less than 75% cells stained) or “homogeneous” (i.e., at least 75% cells stained) instead of “focal” (i.e., greater than 0% and less than 25% cells stained) is also indicative of increased FRα expression.

In some aspects, at least one cell in a cancer sample obtained from a patient has an FRα expression score of at least 1. In some aspects, at least one cell in a cancer sample obtained from a patient has an FRα score of at least 2 (moderate). In some aspects, at least one cell in a cancer sample obtained from a patient has an FRα score of at least 3. In some aspects, the cancer sample is a formalin fixed paraffin embedded sample.

In some aspects, a cancer sample from the patient exhibits increased expression of FRα measured by an immunohistochemistry (IHC) assay that can determine the percentage of cancer cells with moderate (2+) and/or strong (3+) membrane staining compared to the total number of viable tumor cells.

In some aspects, at least 25% of cells in a cancer sample obtained from the patient have an immunohistochemistry (IHC) score of at least 2. In some aspects, at least 75% of cells in a cancer sample obtained from the patient have an immunohistochemistry (IHC) score of at least 2. In some aspects, a positive FRα expression score is defined as ≥75% of viable tumor cells with moderate (2+) and/or strong (3+) membrane staining. In some aspects, a negative FRα expression is defined as <75% of viable tumor cells with moderate (2+) and/or strong (3+) membrane staining.

In some aspects of the methods disclosed herein, the immunoconjugate is administered if the patient's FRα-expressing cancer sample displays a positive FRα expression score as defined by the assay.

In some aspects, the FRα IHC score has been detected prior to the administration. In some aspects the cancer sample used for the IHC determination was obtained at least 6 months, at least 9 months, at least a year, at least 18 months, or at least 2 years prior to the administration. In some aspects, the method further comprises detecting the FRα IHC score prior to the administration. In some aspects, the soluble FRα and the FRα IHC score have been detected prior to the administration. In some aspects the cancer sample used for the IHC determination was obtained at least 6 months, at least 9 months, at least a year, at least 18 months, or at least 2 years prior to the administration.

Immunodetection assay kits for the immunodetection of FRα expression are commercially available. See, e.g., the Ventana FOLR1 (FOLR1-2.1) CDx assay. In some aspects, expression of FRα is measured by IHC and performed using the FOLR1-2.1 detection antibody. The FOLR1-2.1 detection antibody is commercially available as part of the Ventana FOLR1 (FOLR1-2.1) CDx assay.

An antibody or immunoconjugate can be combined in a pharmaceutical combination formulation, or dosing regimen as combination therapy, with a second compound. In some aspects, the second compound is a steroid. In some aspects, the methods encompass administration of a steroid and an immunoconjugate that results in a reduction of ocular changes and/or toxicity as compared to administration of the immunoconjugate alone. In some aspects, the steroid is administered as an eye drop. In some aspects, the steroid comprises 1% prednisolone or difluprednate 0.05%. In some aspects, the method further comprises administering to the subject artificial tears.

The steroid can be administered at the same time as the immunoconjugate, prior to the administration of the immunoconjugate, and/or after the administration of the immunoconjugate. In some aspects, the steroid is administered within about a week, about five days, about three days, about two days, or about one day or 24 hours prior to the administration of the immunoconjugate. In some aspects, the steroid is administered within one day of the administration of the immunoconjugate. In some aspects, the steroid is administered multiple times. In some aspects, the steroid is administered about one day prior to the administration of the immunoconjugate and on the same day as the administration of the immunoconjugate. The steroid can be administered via any number of ways, including for example, topical, pulmonary, oral, parenteral, or intracranial administration. In some aspects, the administration is oral. In some aspects, the administration is intravenous. In some aspects, the administration is both oral and intravenous.

In some aspects of the methods disclosed herein, the patient has a BRCA mutation.

In some aspects of the methods disclosed herein, the patient does not have a BRCA mutation.

In some aspects of the methods disclosed herein, a patient or subject has at least one measurable disease lesion as defined by RECIST v1.1. The methods described herein can result in a decrease in tumor size. The methods described herein can result in a decrease in CA125 levels in ovarian cancer patients. In one example, CA125 levels are measured in a sample from an ovarian cancer patient prior to treatment and then one or more times after treatment, and a decrease in the CA125 level over time is indicative of therapeutic efficacy. The methods described herein can result in an increased time between cancer treatments. The methods described herein can result in increased objective response rate (ORR). The methods described herein can result in increased progression free survival (PFS). The methods described herein can result in increased disease-free survival (DFS). The methods described herein can result in increased overall survival (OS). The methods described herein can result in increased complete response (CR). The methods described herein can result in increased partial response (PR). The methods described herein can result in increased stable disease (SD). The methods described herein can result in increased decrease in progressive disease (PD). The methods described herein can result in a increased time to progression (TTP).

In some aspects, the administration results in a CA125 response rate of about greater than 50%, greater than 55%, greater than 60%, greater than 65%, greater than 70%, greater than 75%, greater than 80%, greater than 85%, or greater than 90%. In some aspects, the administration results in a CA125 response rate between about 60% and about 86%. In some aspects, the administration results in a CA125 response rate of about 75%. In some aspects, the administration results in a CA125 response rate of 74.5%.

In some aspects, the administration results in an objective response rate (ORR) of about greater than 25%, greater than 30%, greater than 35%, greater than 40%, greater than 45%, greater than 50%, greater than 55%, greater than 60%, greater than 65%, or greater than 70% or more. In some aspects, the administration results in an objective response rate (ORR) between 40% and 65%. In some aspects, the administration results in an objective response rate (ORR) of about 52%. In some aspects, the administration results in an objective response rate (ORR) of 51.9%.

In some aspects, the administration results in an duration of response (DOR) of at least 2 months, at least 3 months, at least 4 months, at least 5 months, at least 6 months, at least 7 months, at least 8 months, at least 9 months, at least 10 months, at least 11 months, or at least 12 months. In some aspects, the administration results in an duration of response (DOR) of at least 12 months, at least 13 months, at least 14 months, at least 15 months, at least 16 months, at least 17 months, at least 18 months, at least 19 months, at least 20 months, at least 21 months, at least 22 months, at least 23 months, or at least 24 months. In some aspects, the administration results in an duration of response (DOR) of 4 months. In some aspects, the administration results in an duration of response (DOR) of 5 months. In some aspects, the administration results in an duration of response (DOR) of at least 6 months. In some aspects, the administration results in an duration of response (DOR) of at least 7 months. In some aspects, the administration results in an duration of response (DOR) of at least 8 months. In some aspects, the administration results in an duration of response (DOR) of at least 9 months. In some aspects, the administration results in an duration of response (DOR) of at least 10 months. In some aspects, the administration results in an duration of response (DOR) of 11 months. In some aspects, the administration results in an duration of response (DOR) of 12 months. In some aspects, the administration results in an duration of response (DOR) of 5 to 10 months. In some aspects, the administration results in an duration of response (DOR) of 6 to 10 months. In some aspects, the administration results in an duration of response (DOR) of 6 to 11 months.

In some aspects, administration of the immunoconjugate results in a progression-free survival (PFS) of from at least 1 to at least 12 months. In some aspects, administration of the immunoconjugate results in a progression-free survival (PFS) of from at least 1 to at least 8 months. In some aspects, administration of the immunoconjugate results in a progression-free survival (PFS) of from at least 2 to at least 8 months. In some aspects, administration of the immunoconjugate results in a progression-free survival (PFS) of from at least 2 to at least 6 months. In some aspects, administration of the immunoconjugate results in a progression-free survival (PFS) of from at least 3 to at least 5 months. In some aspects, administration of the immunoconjugate results in a progression-free survival (PFS) of from 1 to 12 months. In some aspects, administration of the immunoconjugate results in a progression-free survival (PFS) of from 1 to 8 months. In some aspects, administration of the immunoconjugate results in a progression-free survival (PFS) of from 2 to 8 months. In some aspects, administration of the immunoconjugate results in a progression-free survival (PFS) of from 2 to 6 months. In some aspects, administration of the immunoconjugate results in a progression-free survival (PFS) of from 3 to 5 months. In some aspects, administration of the immunoconjugate results in a progression-free survival (PFS) of from 6 to 9 months. In some aspects, administration of the immunoconjugate results in a progression-free survival (PFS) of from 5 to 9 months. In some aspects, administration of the immunoconjugate results in a progression-free survival (PFS) of from 6 to 8 months. In some aspects, administration of the immunoconjugate results in a progression-free survival (PFS) of from 5 to 8 months. In some aspects, administration of the immunoconjugate results in a progression-free survival (PFS) of about 4 months. In some aspects, administration of the immunoconjugate results in a progression-free survival (PFS) of about 7 months. In some aspects, administration of the immunoconjugate results in a progression-free survival (PFS) of 3.98 months. In some aspects, administration of the immunoconjugate results in a progression-free survival (PFS) of about 6.93 months. In some aspects, administration of the immunoconjugate results in an about 80% chance of achieving a progression-free survival (PFS) of at least 3 months. In some aspects, administration of the immunoconjugate results in an about 61% chance of achieving a progression-free survival (PFS) of at least 4 months. In some aspects, administration of the immunoconjugate results in an about 39% chance of achieving a progression-free survival (PFS) of at least 6 months. In some aspects, administration of the immunoconjugate results in an about 19% chance of achieving a progression-free survival (PFS) of at least 12 months. In some aspects, the administration of the immunoconjugate increases the patient's overall survival.

In a particular aspect, the methods described herein can result in increased overall survival (OS) of the patient.

In a particular aspect, the method described herein result in a clinical benefit to the patient.

### B. Cytotoxic Agents

The cytotoxic agent in the immunoconjugate can be any compound that results in the death of a cell, or induces cell death, or in some manner decreases cell viability (e.g., In certain aspects, cytotoxic agents useful in the present invention are maytansinoids and maytansinoid analogs. Examples of suitable maytansinoids include esters of maytansinol and maytansinol analogs. Included are any drugs that inhibit microtubule formation and that are highly toxic to mammalian cells, as are maytansinol and maytansinol analogs. Examples of suitable maytansinol esters include those having a modified aromatic ring and those having modifications at other positions. Such suitable maytansinoids are disclosed in U.S. Pat. Nos. 4,424,219; 4,256,746; 4,294,757; 4,307,016; 4,313,946; 4,315,929; 4,331,598; 4,361,650; 4,362,663; 4,364,866; 4,450,254; 4,322,348; 4,371,533; 5,208,020; 5,416,064; 5,475,092; 5,585,499; 5,846,545; 6,333,410; 7,276,497 and 7,473,796.

Exemplary cytotoxic agents were described previously in WO 2018/160539 A1 and WO 2011/106528, each of which is herein incorporated by reference in its entirety.

In some aspects, the immunoconjugate comprises N^{2′}-deacetyl-N^{2′}-(3-mercapto-1-oxopropyl)-maytansine (DM1). In some aspects, the immunoconjugate comprises N^{2′}-deacetyl-N^{2′}-(4-mercapto-4-methyl-1-oxopentyl) maytansine (DM4). DM1 and DM4 were previously described in PCT Application Publication No. WO 2011/106528 A1 and U.S. Pat. No. 8,557,966 B2, each of which is herein incorporated by reference in its entirety.

In some aspects, the immunoconjugate comprises DM4. DM4 is represented by the following structural formula (II):

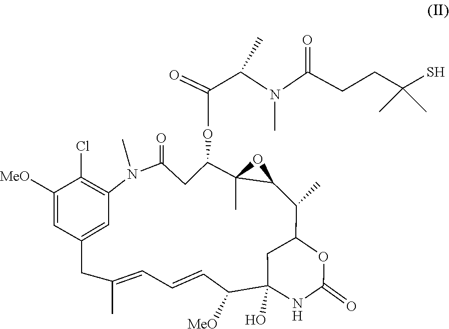 [SMILES: COc1cc2cc(c1Cl)N(C)C(=O)C[C@H](OC(=O)[C@H](C)N(C)C(=O)CCC(C)(C)S)[C@]1(C)O[C@H]1[C@H](C)C1C[C@@](O)(NC(=O)O1)[C@H](OC)/C=C/C=C(\C)C2]

The immunoconjugates can, according to some aspects described herein, be internalized into cells. The immunoconjugate, therefore, can exert a therapeutic effect when it is taken up by, or internalized, by an FRα-expressing cell. In some particular aspects, the immunoconjugate comprises an antibody, antibody fragment, or polypeptide, linked to a cytotoxic agent by a cleavable linker, and the cytotoxic agent is cleaved from the antibody, antibody fragment, or polypeptide, wherein it is internalized by an FRα-expressing cell.

The drug can, for example, be linked to the anti-FRα antibody or fragment thereof through a disulfide bond. The linker molecule or crosslinking agent comprises a reactive chemical group that can react with the anti-FRα antibody or fragment thereof. The reactive chemical groups for reaction with the cell-binding agent can be N-succinimidyl esters and N-sulfosuccinimidyl esters. Additionally the linker molecule comprises a reactive chemical group, which can be a dithiopyridyl group that can react with the drug to form a disulfide bond. Linker molecules include, for example, N-succinimidyl 3-(2-pyridyldithio) propionate (SPDP) (see, e.g., Carlsson et al., Biochem. J., 173: 723-737 (1978)), N-succinimidyl 4-(2-pyridyldithio)butanoate (SPDB) (see, e.g., U.S. Pat. No. 4,563,304), N-succinimidyl 4-(2-pyridyldithio)2-sulfobutanoate (sulfo-SPDB) (see US Publication No. 20090274713), N-succinimidyl 4-(2-pyridyldithio) pentanoate (SPP) (see, e.g., CAS Registry number 341498-08-6), 2-iminothiolane, or acetylsuccinic anhydride. For example, the antibody or cell binding agent can be modified with crosslinking reagents and the antibody or cell binding agent containing free or protected thiol groups thus derived is then reacted with a disulfide- or thiol-containing maytansinoid to produce conjugates. The conjugates can be purified by chromatography, including but not limited to HPLC, size-exclusion, adsorption, ion exchange and affinity capture, dialysis or tangential flow filtration. In some aspects, the linker is selected from the group consisting of: N-succinimidyl 4-(2-pyridyldithio)-2-sulfopentanoate (sulfo-SPP) and N-succinimidyl 4-(2-pyridyldithio)-2-sulfobutanoate (sulfo-SPDB). In a particular aspect, the linker is N-succinimidyl 4-(2-pyridyldithio)-2-sulfobutanoate (sulfo-SPDB).

Several descriptions for producing such antibody-maytansinoid conjugates are provided in U.S. Pat. Nos. 6,333,410, 6,441,163, 6,716,821, and 7,368,565, each of which is incorporated herein in its entirety.

In general, a solution of an antibody in aqueous buffer can be incubated with a molar excess of maytansinoids having a disulfide moiety that bears a reactive group. The reaction mixture can be quenched by addition of excess amine (such as ethanolamine, taurine, etc.). The maytansinoid-antibody conjugate can then be purified by gel filtration.

The number of maytansinoid molecules bound per antibody molecule can be determined by measuring spectrophotometrically the ratio of the absorbance at 252 nm and 280 nm. The average number of maytansinoid molecules/antibody can be, for example, 1-10 or 2-5. The average number of maytansinoid molecules/antibody can be, for example, about 3 to about 4. The average number of maytansinoid molecules/antibody can be about 3.5.

The immunoconjugates can, according to some aspects described herein, be internalized into cells. The immunoconjugate, therefore, can exert a therapeutic effect when it is taken up by, or internalized, by a FRα-expressing cell. In some particular aspects, the immunoconjugate comprises an antibody, antibody fragment, or polypeptide, linked to a cytotoxic agent by a cleavable linker, and the cytotoxic agent is cleaved from the antibody, antibody fragment, or polypeptide, wherein it is internalized by a FRα-expressing cell.

In some aspects of the present disclosure, about 2 to about 8 drug molecules (“drug load”), for example, maytansinoids, are linked to an anti-FRA antibody or fragment thereof. “Drug load”, as used herein, refers to the number of drug molecules (e.g., a maytansinoid) that can be attached to an anti-FRA antibody or fragment thereof. In one aspect, the number of drug molecules that can be attached to a cell binding agent can average from about 2 to about 8 (e.g., 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1). N2′-deacetyl-N2′-(3-mercapto-1-oxopropyl)-maytansine (DM1) and N2′-deacetyl-N2′-(4-mercapto-4-methyl-1-oxopentyl) maytansine (DM4) can be used.

In one aspect, an immunoconjugate (e.g., an immunoconjugate comprising the linker SPDB and the maytansinoid DM4) comprises about 1 to about 8 maytansinoids per antibody. In another aspect, an immunoconjugate (e.g., an immunoconjugate comprising the linker SPDB and the maytansinoid DM4) comprises about 2 to about 7 maytansinoids per antibody. In another aspect, an immunoconjugate (e.g., an immunoconjugate comprising the linker SPDB and the maytansinoid DM4) comprises about 2 to about 6 maytansinoids per antibody. In another aspect, an immunoconjugate (e.g., an immunoconjugate comprising the linker SPDB and the maytansinoid DM4) comprises about 2 to about 5 maytansinoids per antibody. In another aspect, an immunoconjugate (e.g., an immunoconjugate comprising the linker SPDB and the maytansinoid DM4) comprises about 3 to about 5 maytansinoids per antibody. In another aspect, an immunoconjugate (e.g., an immunoconjugate comprising the linker SPDB and the maytansinoid DM4) comprises about 3 to about 4 maytansinoids per antibody.

In one aspect, a composition comprising immunoconjugates has an average of about 2 to about 8 (e.g., 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1) drug molecules (e.g., maytansinoids) attached per antibody. In one aspect, a composition comprising immunoconjugates has an average of about 1 to about 8 drug molecules (e.g., maytansinoids) per antibody. In one aspect, a composition comprising immunoconjugates has an average of about 2 to about 7 drug molecules (e.g., maytansinoids) per antibody. In one aspect, a composition comprising immunoconjugates has an average of about 2 to about 6 drug molecules (e.g., maytansinoids) per antibody. In one aspect, a composition comprising immunoconjugates has an average of about 2 to about 5 drug molecules (e.g., maytansinoids) per antibody. In one aspect, a composition comprising immunoconjugates has an average of about 3 to about 5 drug molecules (e.g., maytansinoids) per antibody. In one aspect, a composition comprising immunoconjugates has an average of about 3 to about 4 drug molecules (e.g., maytansinoids) per antibody.

In one aspect, a composition comprising immunoconjugates has an average of about 2±0.5, about 3±0.5, about 4±0.5, about 5±0.5, about 6±0.5, about 7±0.5, or about 8±0.5 drug molecules (e.g., maytansinoids) attached per antibody. In one aspect, a composition comprising immunoconjugates has an average of about 3.5±0.5 drug molecules (e.g., maytansinoids) per antibody.

As used herein, the “linked to an anti-FRα antibody or fragment thereof” refers to the conjugate molecule comprising at least one drug derivative bound to a cell-binding agent, anti-FRα antibody, or fragment via a suitable linking group, or a precursor thereof. An exemplary linking group is sulfo-SPDB.

### C. Linkers

Any suitable linkers known in the art can be used in preparing the immunoconjugates. In some aspects, the linkers are bifunctional linkers. As used herein, the term “bifunctional linker” refers to modifying agents that possess two reactive groups; one of which is capable of reacting with a cell binding agent while the other one reacts with the maytansinoid compound to link the two moieties together. Such bifunctional crosslinkers are well known in the art (see, for example, Isalm and Dent in Bioconjugation chapter 5, p218-363, Groves Dictionaries Inc. New York, 1999). For example, bifunctional crosslinking agents that enable linkage via a thioether bond include N-succinimidyl-4-(N-maleimidomethyl)-cyclohexane-1-carboxylate (SMCC) to introduce maleimido groups, or with N-succinimidyl-4-(iodoacetyl)-aminobenzoate (SIAB) to introduce iodoacetyl groups. Other bifunctional crosslinking agents that introduce maleimido groups or haloacetyl groups on to a cell binding agent are well known in the art (see US Patent Publication Nos. 2008/0050310, 20050169933, available from Pierce Biotechnology Inc. P.O. Box 117, Rockland, IL 61105, USA) and include, but not limited to, bis-maleimidopolyethyleneglycol (BMPEO), BM(PEO)₂, BM(PEO)₃, N-(β-maleimidopropyloxy)succinimide ester (BMPS), γ-maleimidobutyric acid N-succinimidyl ester (GMBS), ε-maleimidocaproic acid N-hydroxysuccinimide ester (EMCS), 5-maleimidovaleric acid NHS, HBVS, N-succinimidyl-4-(N-maleimidomethyl)-cyclohexane-1-carboxy-(6-amidocaproate), which is a “long chain” analog of SMCC (LC-SMCC), m-maleimidobenzoyl-N-hydroxysuccinimide ester (MBS), 4-(4-N-maleimidophenyl)-butyric acid hydrazide or HCl salt (MPBH), N-succinimidyl 3-(bromoacetamido)propionate (SBAP), N-succinimidyl iodoacetate (SIA), κ-maleimidoundecanoic acid N-succinimidyl ester (KMUA), N-succinimidyl 4-(p-maleimidophenyl)-butyrate (SMPB), succinimidyl-6-(β-maleimidopropionamido)hexanoate (SMPH), succinimidyl-(4-vinylsulfonyl)benzoate (SVSB), dithiobis-maleimidoethane (DTME), 1,4-bis-maleimidobutane (BMB), 1,4-bismaleimidyl-2,3-dihydroxybutane (BMDB), bis-maleimidohexane (BMH), bis-maleimidoethane (BMOE), sulfosuccinimidyl 4-(N-maleimido-methyl)cyclohexane-1-carboxylate (sulfo-SMCC), sulfosuccinimidyl(4-iodo-acetyl)aminobenzoate (sulfo-SIAB), m-maleimidobenzoyl-N-hydroxysulfosuccinimide ester (sulfo-MBS), N-(γ-maleimidobutryloxy)sulfosuccinimide ester (sulfo-GMBS or sGMBS), N-(ε-maleimidocaproyloxy)sulfosuccimido ester (sulfo-EMCS), N-(κ-maleimidoundecanoyloxy)sulfosuccinimide ester (sulfo-KMUS), and sulfosuccinimidyl 4-(p-maleimidophenyl)butyrate (sulfo-SMPB).

Heterobifunctional crosslinking agents are bifunctional crosslinking agents having two different reactive groups. Heterobifunctional crosslinking agents containing both an amine-reactive N-hydroxysuccinimide group (NHS group) and a carbonyl-reactive hydrazine group can also be used to link the cytotoxic compounds described herein with an anti-FRα antibody or antigen-binding fragment thereof. Examples of such commercially available heterobifunctional crosslinking agents include succinimidyl 6-hydrazinonicotinamide acetone hydrazone (SANH), succinimidyl 4-hydrazidoterephthalate hydrochloride (SHTH) and succinimidyl hydrazinium nicotinate hydrochloride (SHNH). Conjugates bearing an acid-labile linkage can also be prepared using a hydrazine-bearing benzodiazepine derivative of the present disclosure. Examples of bifunctional crosslinking agents that can be used include succinimidyl-p-formyl benzoate (SFB) and succinimidyl-p-formylphenoxyacetate (SFPA).

In particular aspects, the linker of the present disclosure is N-succinimidyl 4-(2-pyridyldithio)-2-sulfobutanoate (sulfo-SPDB).

Aspects of the present disclosure can be further defined by reference to the following non-limiting examples, which describe in detail preparation of certain antibodies of the present disclosure and methods for using antibodies of the present disclosure. It will be apparent to those skilled in the art that many modifications, both to materials and methods, can be practiced without departing from the scope of the present disclosure.

### Example

It is understood that the examples and aspects described herein are for illustrative purposes only and that various modifications or changes in light thereof will be suggested to persons skilled in the art and are to be included within the spirit and purview of this application.

A Phase 2, Single Arm Study of Mirvetuximab Soravtansine in Recurrent Platinum Sensitive, High-Grade Epithelial Ovarian, Primary Peritoneal, or Fallopian Tube Cancers with High Folate Receptor-Alpha Expression

In this Phase 2, Single Arm Study, 75 patients, at approximately 75 centers globally, were studied for about 30 months. This Phase 2 study was designed to evaluate the efficacy and safety of Mirvetuximab Soravtansine (i.e., MIRV or IMGN853) in patients with recurrent platinum-sensitive, high-grade serous epithelial ovarian cancer (EOC), primary peritoneal, or fallopian tube cancer, whose tumors express a high-level of FRα, referred to throughout as rPSOC (recurrent platinum-sensitive ovarian cancer). Patients had at least 2 prior lines of therapy. These included at least 2 lines of platinum-containing therapy or 1 line with a documented platinum allergy. All patients were appropriate for non-platinum single-agent therapy for their next line of therapy. FRα positivity was defined by the Ventana FRA (FRA-2.1) CDx assay (Ventana FRA Assay). Seventy-nine (79) patients were enrolled. Efficacy evaluable patients have at least 1 measurable lesion (per Response Evaluation Criteria in Solid Tumors version 1.1 [RECIST v1.1]) at baseline and received at least 1 dose of MIRV. All patients received a single-agent MIRV at 6 mg/kg adjusted ideal body weight (AIBW) administered through intravenous (IV) infusion on Day 1 of every 3-week cycle (Q3W). Tumor response was evaluated using RECIST v1.1. Computerized tomography (CT) or magnetic resonance imaging (MRI) scans were collected for sensitivity analysis by BICR.

Patients continued to receive MIRV until PD, unacceptable toxicity, withdrawal of consent, death, or until the study was terminated (whichever occured first).

Tumor assessments, including radiological assessments by CT/MRI scans, were performed at screening and subsequently every 6 weeks (±1 week) from Cycle 1 Day 1 (C1D1) for the first 36 weeks then every 12 weeks (±3 weeks) until PD, death, the start of new anticancer therapy, or patient's withdrawal of consent (whichever occured first).

Patients who discontinued MIRV for reasons other than PD continued with tumor assessments until documentation of PD or a new anticancer therapy was started, whichever came first. Prior to Week 36 (from C1D1), assessments occured every 6 weeks (±1 week) as allowed by local requirements but at an interval of no more than 12 weeks. After Week 36, assessments occured every 12 weeks (±3 weeks) until PD per RECIST v1.1 was documented or a new anticancer therapy was started.

All patients who discontinued MIRV were followed for survival every 3 months (±1 month) until death, lost to follow-up, withdrawal of consent for survival follow-up, or End of Study (EOS) (whichever came first). Additional survival follow-up calls occured periodically.

### Primary Objective

to determine the efficacy of Mirvetuximab Soravtansine (i.e., MIRV, or IMGN853) in patients with recurrent platinum-sensitive ovarian cancer (rPSOC) and high folate receptor alpha (FRα) expression.

### Secondary Objective

to determine the durability of response to MIRV in patients with rPSOC and high FRα expression.

### Additional Secondary Objectives

to evaluate the safety and tolerability of MIRV and to characterize the clinical activity of MIRV in patients with rPSOC and high FRα expression.

### Primary Endpoint

Objective response rate (ORR), which includes confirmed best response of complete response (CR) or partial response (PR) as assessed by the Investigator.

### Key Secondary Endpoint

Duration of response (DOR), defined as the time from initial Investigator-assessed response (CR or PR) until progressive disease (PD) as assessed by the Investigator

### Additional Secondary Endpoints

Treatment-emergent adverse events (TEAEs) and laboratory test results, physical examination, or vital signs
CA-125 response determined using the Gynecologic Cancer Intergroup (GCIG) criteria
Progression-free survival (PFS), defined as the time from first dose of MIRV until Investigator-assessed radiological PD or death, whichever occurs first
Overall survival (OS), defined as the time from first dose of MIRV until death
ORR, DOR, and PFS by blinded independent central review (BICR) will be summarized as sensitivity analysis

### INCLUSION CRITERIA

1. Patients must be ≥18 years of age at the time of screening.
2. Patients must have had an Eastern Cooperative Oncology Group Performance Status (ECOG PS) of 0 or 1
3. Patients must have had a confirmed diagnosis of high-grade serous EOC, primary peritoneal cancer, or fallopian tube cancer
4. Patients must have had platinum-sensitive disease defined as radiographic progression greater than 6 months from last dose of most recent platinum therapy
5. Patients must have had progressed radiographically on or after their most recent line of anticancer therapy
6. Patients must have had at least 1 lesion that meets the definition of measurable disease by RECIST v1.1
7. Patients must have been willing to provide an archival tumor tissue block or slides, or undergo procedure to obtain a new biopsy using a low-risk, medically routine procedure for immunohistochemistry (IHC) confirmation of FRα positivity
8. Patient's tumor must have been positive for FRα expression as defined by the Ventana FRA Assay
9. Prior anticancer therapy
   a. Patients must have received at least 2 prior systemic lines of platinum therapy and have been considered by the Investigator as appropriate for single-agent non-platinum therapy (documentation required—e.g., high risk of hypersensitivity reaction; risk of further cumulative toxicity with additional platinum, including but not limited to myelosuppression, neuropathy, renal insufficiency or other) Note: Patients who have had a documented platinum allergy may have had only 1 prior line of platinum
   b. Patients may have received up to but no more than 1 prior independent non-platinum cytotoxic therapy
   c. Patients must have had testing for breast cancer susceptibility gene (BRCA) mutation (tumor or germline) and, if positive, must have received a prior poly (ADP-ribose) polymerase (PARP) inhibitor as either treatment or maintenance therapy
   d. Neoadjuvant t adjuvant therapies were considered 1 line of therapy
   e. Maintenance therapy (e.g., bevacizumab, PARP inhibitors) was considered part of the preceding line of therapy (i.e., not counted independently)
   f. Therapy changed due to toxicity in the absence of progression was considered part of the same line (i.e., not counted independently)
10. Patients must have completed prior therapy within the specified times below:
   a. Systemic antineoplastic therapy within 5 half-lives or 4 weeks (whichever is shorter) prior to first dose of MIRV
   b. Focal radiation completed at least 2 weeks prior to first dose of MIRV
11. Patients must have stabilized or recovered (Grade 1 or baseline) from all prior therapy-related toxicities (except alopecia)
12. Patients must have completed any major surgery at least 4 weeks prior to first dose of MIRV and have recovered or stabilized from the side effects of prior surgery prior to first dose of MIRV
13. Patients must have had adequate hematologic, liver and kidney functions defined as:
   a. Absolute neutrophil count (ANC)≥1.5×10⁹/L (1500/μL) without granulocyte colony-stimulating factor (G-CSF) in the prior 10 days or long-acting white blood cell (WBC) growth factors in the prior 20 days
   b. Platelet count ≥100×10⁹/L (100,000/μL) without platelet transfusion in the prior 10 days
   c. Hemoglobin ≥9.0 g/dL without packed red blood cell (PRBC) transfusion in the prior 21 days
   d. Serum creatinine ≤1.5× upper limit of normal (ULN)
   e. Aspartate aminotransferase (AST) and alanine aminotransferase (ALT)≤3.0×ULN
   f. Serum bilirubin ≤1.5×ULN (patients with documented diagnosis of Gilbert syndrome are eligible if total bilirubin <3.0×ULN)
   g. Serum albumin ≥2 g/dL
14. Patients must have been willing and able to sign the informed consent form (ICF) and to adhere to the protocol requirements
15. Women of childbearing potential (WCBP) must have agreed to use highly effective contraceptive method(s) while on MIRV and for at least 3 months after the last dose
16. WCBP must have had a negative pregnancy test within the 4 days prior to the first dose of MIRV

### EXCLUSION CRITERIA

1. Patients with endometrioid, clear cell, mucinous, or sarcomatous histology, mixed tumors containing any of the above histologies, or low-grade/borderline ovarian tumor
2. Patients with prior wide-field radiotherapy (RT) affecting at least 20% of the bone marrow
3. Patients with >Grade 1 peripheral neuropathy per Common Terminology Criteria for Adverse Events (CTCAE)
4. Patients with active or chronic corneal disorders, history of corneal transplantation, or active ocular conditions requiring ongoing treatment/monitoring, such as uncontrolled glaucoma, wet age-related macular degeneration requiring intravitreal injections, active diabetic retinopathy with macular edema, macular degeneration, presence of papilledema, and/or monocular vision
5. Patients with serious concurrent illness or clinically relevant active infection, including, but not limited to the following:
   a. Active hepatitis B or C infection (whether or not on active antiviral therapy)
   b. HIV infection
   c. Active cytomegalovirus infection
   d. Any other concurrent infectious disease requiring IV antibiotics within 2 weeks prior to the first dose of MIRV
   Note: Testing at screening is not required for the above infections unless clinically indicated.
6. Patients with a history of multiple sclerosis (MS) or other demyelinating disease and/or Lambert-Eaton syndrome (paraneoplastic syndrome)
7. Patients with clinically significant cardiac disease including, but not limited to, any of the following:
   a. Myocardial infarction ≤6 months prior to first dose
   b. Unstable angina pectoris
   c. Uncontrolled congestive heart failure (New York Heart Association >class II)
   d. Uncontrolled≥Grade 3 hypertension (per CTCAE)
   e. Uncontrolled cardiac arrhythmias
8. Patients with a history of hemorrhagic or ischemic stroke within 6 months prior to enrollment
9. Patients with a history of cirrhotic liver disease (Child-Pugh Class B or C)
10. Patients with a previous clinical diagnosis of noninfectious interstitial lung disease (ILD), including noninfectious pneumonitis
11. Patients requiring use of folate-containing supplements (e.g., folate deficiency)
12. Patients with prior hypersensitivity to monoclonal antibodies (mAb)
13. Women who are pregnant or breastfeeding
14. Patients who received prior treatment with MIRV or other FRα-targeting agents
15. Patients with untreated or symptomatic central nervous system (CNS) metastases
16. Patients with a history of other malignancy within 3 years prior to enrollment Note: patients with tumors with a negligible risk for metastasis or death (e.g., adequately controlled basal-cell carcinoma or squamous-cell carcinoma of the skin, or carcinoma in situ of the cervix or breast) were eligible.
17. Prior known hypersensitivity reactions to study drugs and/or any of their excipients Prospective approval of protocol deviations to recruitment and enrollment criteria, also known as protocol waivers or exemptions, was not permitted.

The study was designed to test the null hypothesis that the ORR is ≤28% using an optimal Simon's two-stage design (Simon 1989) without a planned pause in enrollment given preliminary efficacy and established safety of mirvetuximab soravtansine. All patients received single-agent mirvetuximab soravtansine at 6 mg/kg AIBW administered through IV infusion on Day 1 of every 3-week cycle (Q3W). Computerized tomography (CT) or magnetic resonance imaging (MRI) scans were collected for sensitivity analysis by blinded independent central review (BICR). Patients continued to receive mirvetuximab soravtansine until PD, unacceptable toxicity, withdrawal of consent, death, or until the study was terminated, whichever occurred first.

Tumor assessments, including radiological assessment by CT/MRI scans, were performed at screening and subsequently every 6 weeks (±1 week) from Cycle 1 Day 1 (C1D1) for the first 36 weeks then every 12 weeks (±3 weeks) until PD, death, the start of new anticancer therapy, or patient's withdrawal of consent, whichever occurred first.

Patients who discontinued mirvetuximab soravtansine for reasons other than progressive disease (PD) continued with tumor assessments until documentation of PD or the start of a new anticancer therapy, whichever came first. Prior to Week 36 (from C1D1), assessments occured every 6 weeks (±1 week) as allowed by local requirements but must have occured at an interval of no more than 12 weeks. After week 36, assessments occured every 12 weeks (±3 weeks) until documentation of PD per RECIST v1.1 or the start of new anticancer therapy.

All patients who discontinued mirvetuximab soravtansine were followed for survival every 3 months (±1 month) until death, lost to follow-up, withdrawal of consent for survival follow-up, or End of Study (EOS), whichever came first. Additional survival follow-up calls occured periodically, if needed.

All patients must have received 325 to 650 mg of acetaminophen/paracetamol (orally [PO] or IV), 10 mg IV dexamethasone, and 25 to 50 mg diphenhydramine (IV or PO) (equivalent drugs of similar drug classes was also acceptable) approximately 30 minutes before each infusion of mirvetuximab soravtansine. If individual patients required more intensive treatment to prevent infusion-related reactions (IRRs), investigators modified the regimen accordingly. An antiemetic medication (e.g., 5-HT3 serotonin receptor antagonists such as palonosetron, granisetron or ondansetron or appropriate alternatives) was recommended before each mirvetuximab soravtansine dose and could be used any time at the discretion of the treating physician.

All patients were mandated to use corticosteroid eye drops as prescribed by the treating physician unless the risk outweighed the benefit as per the ophthalmologist/physician. All patients were instructed to self-administer 1% prednisolone (Pred Forte® or generic equivalent) 6 times daily on Days −1 to 4 and 4 times daily (QID) on Days 5 to 8 of each cycle during the study. For individual patients who could not tolerate the preservative contained in 1% prednisolone, other corticosteroid eye drops were substituted (e.g., difluprednate 0.05%; Durezol®) and administered on Days −1 to 8 of each cycle at a frequency prescribed by the ophthalmologist. If prednisolone eye drops could not be obtained, alternate steroid eye drops were acceptable.

Patients were mandated to use lubricating artificial tears on a daily basis (as directed by the product label or the treating physician). Preservative-free lubricating drops were recommended. Patients were advised to wait at least 15 minutes after corticosteroid eye drop administration before instilling lubricating eye drops.

The total dose of mirvetuximab soravtansine was calculated based on each patient's AIBW using the following formula:

$$Adjusted Ideal Body Weight ( A I B W )$$

$$A I B W = I B W 1 + 0.4 ( Actual weight - I B W 1 )$$

$$Where :$$

$$Ideal Body Weight ( I B W )$$

$$I B W 1 ( female ) = 0.9 H 1 - 92$$

$$1 H = height in cm ; W = weight in kg )$$

The weight used for calculation was obtained before study drug administration on C1D1 (14 days). Dosing was modified per institutional standard of care for changes in body weight on subsequent cycles. Dosing were modified for significant (≥10%) changes in body weight (not influenced by weight gain or loss attributed to fluid retention).

Mirvetuximab soravtansine was administered at 6 mg/kg AIBW as an IV infusion following preparation as outlined in the Pharmacy Manual.

At C1D1 mirvetuximab soravtansine was administered at a rate of 1 mg/min; after 30 min, the rate could be increased to 3 mg/min if well tolerated. If well tolerated after 30 min at 3 mg/min, the infusion rate could be increased to 5 mg/min. Subsequent infusions could be delivered at the tolerated rate. Therefore, the overall length of infusion varied depending on dose and patient tolerability. After infusion, the IV line was flushed with 5% dextrose as needed (PRN) to ensure delivery of the full dose.

Patients were carefully observed during each infusion and vital signs are taken as outlined in the Schedule of Assessments. Patients remained in the clinic under observation for 4 hours after the first infusion, and for at least 1 hour after each subsequent infusion. While in the treatment area, patients were closely monitored for AEs.

In the absence of a treatment emergent adverse event (TEAE) that required dose modification a patient must have met the following criteria to receive mirvetuximab soravtansine at any cycle:
ANC must be ≥1.5×10⁹/L (1,500/μL)
Platelet count must be ≥100×109/L (100,000/μL)
All non-hematologic toxicities for which a causal association to study drug cannot be ruled out, must be ≤Grade 2 or returned to baseline; the exceptions to this rule being:
Treatment-emergent ocular disorders, which must have recovered to <Grade 1 or baseline
Treatment emergent pneumonitis which must have recovered to ≤Grade 1

Treatment was discontinued in the case of the following treatment-related events:
≥Grade 3 cardiac event (excluding Grade 3 hypertension)
≥Grade 3 pneumonitis event
Non-hematologic events of Grade 4 severity
Ocular events of Grade 4 severity

Failure to meet re-treatment criteria within 1 cycle (21 days) after the missed dose due to insufficient recovery from a treatment-related toxicity unless otherwise specified in the management guidance for a particular toxicity. In such cases, continuation of administration was considered for those patients who had experienced clinical benefit if agreed upon between the Sponsor and the Investigator.

Patients continued to receive mirvetuximab soravtansine until they presented with PD per RECIST v1.1, as assessed by study Investigator, unacceptable toxicity, withdrew consent, or death, whichever came first, or until the study was terminated. Study treatment and/or participation in the study could be discontinued at any time at the discretion of the Investigator. The following are reasons that the Investigator could remove a patient from the study drug:
The patient suffers an intolerable AE
Noncompliance, including failure to appear at one or more study visits
The patient was erroneously included in the study
   The reason for treatment discontinuation must be captured in the clinical trial database. Any AEs experienced up to the point of discontinuation and 30 days thereafter were documented. All serious adverse events (SAEs), and those AEs assessed by the Investigator as at least possibly related to study drug were followed until they resolved or stabilized. Patients continue to be followed for OS, after discontinuing study drug.

Discontinuation from participation in the study was documented in the clinical trial database. Reasons for EOS included withdrawal of consent, lost to follow-up, death, or study termination by Sponsor.

FRα expression varies with tumor histology, as reported in the literature and demonstrated in preclinical studies. FRα expression in tumor samples were analyzed using the Ventana FOLR1 Assay. This assay was conducted at a central laboratory. All patients submitted tumor tissue block or fresh cut slides from available block (FFPE slides) for analysis of FRα expression prior to enrollment.

To be considered positive for FRα expression and eligibility for the study, ≥75% of viable tumor cells must exhibit level 2 and/or 3 membrane staining intensity. Only patients with the required FRα expression levels by Ventana FOLR1 Assay were eligible to enroll in the study. If a patient wished to enroll and did not have archival material available for analysis, she must have undergone a biopsy to assess FRα expression. Patients for whom the only sites of disease required biopsy procedures considered to be of significant risk were not enrolled in the study. These procedures include (but are not limited to) biopsies of the brain, lung/mediastinum, pancreas, or endoscopic procedures extending beyond the esophagus, stomach, or bowel.

An inclusion criterion for this trial was the BRCA status of patients. This was determined by local testing prior to enrollment.

### Adverse Events

An AE is any noxious, pathologic, or unintended change in anatomical, physiologic, or metabolic function as indicated by physical signs, symptoms, or laboratory changes occurring in any phase of a clinical study, whether or not considered study drug-related. This includes an exacerbation of a pre-existing condition.

AEs include:
Worsening (change in nature, severity, or frequency) of conditions present at the onset of the study
Intercurrent illnesses
Drug interactions
Events related to or possibly related to concomitant medications
Abnormal laboratory values (this includes significant shifts from baseline within the range of normal that the Investigator considers to be clinically important)
Clinically significant abnormalities in PE, vital signs, and weight Serious Adverse Events

An SAE is any AE occurring at any dose that results in any of the following outcomes:
Death
Is life-threatening
Requires inpatient hospitalization
Requires prolongation of existing hospitalization
A persistent or significant disability or incapacity or substantial disruption of the ability to conduct normal life functions
A congenital anomaly or birth defect

Important medical events that may not result in death, be life-threatening, or require hospitalization may be considered an SAE when, based upon appropriate medical judgment, they may jeopardize the patient and may require medical or surgical intervention to prevent one of the outcomes listed in this definition. Examples of such medical events include allergic bronchospasm requiring intensive treatment in an emergency room or at home, blood dyscrasias or convulsions that do not result in inpatient hospitalization

Hospitalization is defined as admission to treat a clinical AE. The following events would not be considered hospitalizations for SAE reporting purposes: 23-hour hold for observation, admission to a hospice facility or nursing home, respite care, outpatient surgery, social admission (e.g., a homeless patient) or admission not associated with a precipitating clinical AE (e.g., elective or pre-planned surgery, or in-patient administration of subsequent chemotherapy, etc.).

All AEs were evaluated according to the NCI CTCAE v5.0

All statistical analyses were performed using SAS statistical software Version 9.4 or later. A detailed description of patient disposition, patient demographics, and baseline characteristics are provided herein.

The primary endpoint was ORR, which includes confirmed best response of CR or PR.

The primary endpoint of ORR (objective response=CR+PR) was estimated along with a 95% CI by the Clopper-Pearson method based on the efficacy evaluable population.

The primary analysis of ORR was conducted after all efficacy evaluable patients had undergone at least 4 post-baseline tumor assessments, o experienced radiological PD, or died or had clinical progression within 105 days of first dose of study drug, or withdrawn from study for other reasons

The key secondary endpoint of Investigator-assessed DOR was estimated using Kaplan-Meier method for survival function estimate.

The initial analysis of DOR was conducted at the time of primary analysis of ORR. An updated analysis of DOR was conducted 12 months after the primary analysis of ORR, at the end of the study.

The additional secondary endpoints of Investigator-assessed PFS and OS were estimated using Kaplan-Meier method for survival function estimate and CA-125 response was determined using the GCIG criteria.

### RESULTS

In sum, most patients in the study had prior bevacizumab, PARPi, and/or taxane therapy, and most patients had lines of prior therapy. 81% of patients had prior PARPi (74.7% of whom had progressed while on a PARPi); 64.6% of patients had prior bevacizumab; 97.5% of patients had prior taxanes, and 98.8% had 2+ prior lines of therapy. The ORR was 51.9%, including 6 complete and 35 partial responses. The best overall response in the safety population was 7.6% CR, 44.3% PR, 36.7% SD. The key secondary endpoint, DOR, was 8.25 months and the PFS was 6.93 months. The ORR prior lines of therapy was: 1-2, 55.1%; 3, 50%; 4+, 33.3%. The ORR with no prior PARPi therapy was 75%. The ORR prior bevacizumab therapy was 49%, while the ORR no prior bevacizumab therapy 57.1%. Discontinuation reasons included pneumonitis (4%); cataract (3%); corneal epithelial microcysts (3%); peripheral neuropathy (3%); blurred vision (3%); cystitis (1%); interstitial lung disease (ILD) (1%); keratopathy (1%). The most common treatment-emergent adverse events (TEAEs) (all grade and grade ≥3) were blurred vision (63% and 10⁰/), dry eye (37% and 3%), nausea (37% and 1%), keratopathy (33% and 4%), and diarrhea (30% and 3%). TEAEs led to dose delays, reductions, and discontinuations in 61%, 42%, and 16% of patients, respectively.

The number of patients and safety population are shown in Table 7.

TABLE 7
Safety Population.

| Number of Patients, n (%) | Total (N = 79) |
|---|---|
| Screened | 302 |
| Treated (Safety Population) [1] | 79 |
| Patient still on Treatment | 11 (14) |
| Discontinued from Treatment | 68 (86) |
| Primary Reason for Treatment Discontinuation | |
| Adverse Event | 13 (16) |
| Death | 2 (3) |
| PI Discretion | 0 |
| Patient Withdrew Consent to Treatment (continue | 0 |
| following the patient) | |
| Patient Withdrew Consent to Treatment and | 0 |
| Follow-Up | |
| Progressive Disease | 53 (67) |
| Protocol Deviation | 0 |
| Study Terminated by Sponsor | 0 |
| Other | 0 |
| Discontinued from Treatment, Entered Response | 18 (23) |
| Follow-up [2] | |
| Discontinued from Treatment, Entered Survival | 49 (62) |
| Follow-up [2] | |

[1] Number of treated patients (Safety Population) will be used as the denominator for the percentages in all subsequent rows.
[2] Patients who have discontinued from study treatment but have not discontinued from study yet.

The baseline characteristics are shown in Table 8. Primary platinum-free interval is the time from last dose of the first platinum therapy to the date of disease progression and/or relapse (recurrence) following that line of therapy (time rounded to whole number). Platinum-free interval is the time from last dose of the latest line platinum therapy to the date of disease progression and/or relapse following that line of therapy (time rounded to whole number). Note: if the patient had progression within 30 days after the last dosing of PARP inhibitor or progression was listed as the reason for treatment discontinuation of PARP inhibitor, the patient was defined as progression on prior PARP inhibitor. If neither, the patient was defined as no progression.

TABLE 8
Baseline Characteristics

| | Total (N = 79) | |
|---|---|---|
| Age (years)[1] | | |
| n | 79 | |
| Mean (SD) | 64.1 | (10.16) |
| Median | 66.0 | |
| Min, Max | 41, 84 | |
| Age Group, n (%) | | |
| 18-64 | 33 | (41.8) |
| >=65 | 46 | (58.2) |
| Primary diagnosis, n (%) | | |
| Epithelial Ovarian | 67 | (84.8) |
| Fallopian Tube | 5 | (6.3) |
| Primary Peritoneal | 4 | (5.1) |
| Other | 3 | (3.8) |
| Any BRCA Mutations, n (%) | | |
| Positive | 22 | (27.8) |
| BRCA1 | 18 | (22.8) |
| BRCA2 | 6 | (7.6) |
| Negative/Unknown | 57 | (72.2) |
| FRα Expression (Percent Staining at | | |
| Intensity 2+), n (%) | | |
| >=75% | 79 | (100) |
| >=90% | 35 | (44.3) |
| >=50%-<75% | 0 | |
| >=25%-<50% | 0 | |
| <25% | 0 | |
| Prior Radiation Therapy, n (%) | | |
| Yes | 9 | (11.4) |
| No | 70 | (88.6) |
| Number of Prior Lines of Systemic | | |
| Therapy, n (%) [2] | | |
| 1 | 1 | (1.3) |
| 2 | 48 | (60.8) |
| 3 | 24 | (30.4) |
| >3 | 6 | (7.6) |
| Prior Exposure to PARP Inhibitors, | | |
| n (%) | | |
| Yes | 64 | (81.0) |
| Progression [3] | 59 | (74.7) |
| No progression [3] | 5 | (6.3) |
| No | 12 | (15.2) |
| Uncertain [4] | 3 | (3.8) |
| Prior Exposure to Bevacizumab, | | |
| n (%) | | |
| Yes | 51 | (64.6) |
| Exposed in 1L only | 24 | (30.4) |
| Exposed in 2L only | 13 | (16.5) |
| Exposed in >=3L only | 7 | (8.9) |
| Exposed in Multiple Lines | 7 | (8.9) |
| No | 28 | (35.4) |
| Primary platinum-free interval | | |
| (Months) [5] | | |
| ≤12 | 16 | (20.3) |
| >12 | 59 | (74.7) |
| Missing | 4 | (5.1) |
| Platinum-free interval (Months) [6] | | |
| ≤12 | 43 | (54.4) |
| >12 | 34 | (43.0) |
| Missing | 2 | (2.5) |
| Prior Exposure to Taxanes, n (%) | | |
| Yes | 77 | (97.5) |
| Exposed in 1L only | 57 | (72.2) |
| Exposed in 2L only | 0 | |
| Exposed in >=3L only | 0 | |
| Exposed in Multiple Lines | 20 | (25.3) |
| No | 2 | (2.5) |
| Prior Exposure to Doxorubicin, | | |
| n (%) | | |
| Yes | 54 | (68.4) |
| No | 25 | (31.6) |
| Prior Exposure to Topotecan, n (%) | | |
| Yes | 0 | |
| No | 79 | (100) |

[1]Only collected age is used for summarization due to local regulations of prohibition for collection of a complete date of birth.
[2] The number of prior systemic therapies is defined as the number of distinct treatment regimens.
[3] If the patient had progression within 30 days after the last dosing of PARP inhibitor or progression is listed as the reason for treatment discontinuation of PARP inhibitor, the patient will be defined as progression on prior PARP inhibitor. If neither, the patient will be defined as no progression.
[4] For patients who participated in double blind trials evaluating PARPi vs placebo and the actual treatment was not known.
[5] Primary platinum-free interval is defined as time from last dose of 1st line platinum therapy to the date of disease progression and/or relapse following 1st line therapy (time rounded to whole number).
[6] Platinum-free interval is defined as time from last dose of the latest line platinum therapy to the date of disease progression and/or relapse following that line of therapy (time rounded to whole number).

The overall response rate (ORR) data (a primary endpoint is shown in Table 9.

TABLE 9
Primary Endpoint: ORR

| | Total (N = 79) | |
|---|---|---|
| Confirmed Best Overall Response, | | |
| n (%) | | |
| Complete Response | 6 | (7.6) |
| Partial Response | 35 | (44.3) |
| Stable Disease | 29 | (36.7) |
| Progressive Disease | 7 | (8.9) |
| Not Evaluable | 2 | (2.5) |
| Objective Response Rate, n (%) [1] | 41 | (51.9) |

| | |
|---|---|
| 95% Exact CI [2] | (40.4, 63.3) |

INV = Investigator;
CI = confidence interval;
[1] Objective response rate is defined as proportion of patients with a confirmed best overall response of complete or partial response.
[2] 95% exact confidence interval is estimated by Clopper-Pearson method, Clopper-Pearson Exact CI.

Concordance of best overall response is shown in Table 10.

TABLE 10
Concordance of Best Overall Response

| | Best Response by BICR | | | |
|---|---|---|---|---|
| Best Response by INV | Responder | Non-Responder | Total | |
| Responder | 26 (32.9) | 15 (19.0) | 41 | (51.9) |
| Non-Responder | 6 (7.6) | 32 (40.5) | 38 | (48.1) |
| Total | 32 (40.5) | 47 (59.5) | 79 | (100.0) |
| Concordant | | | 58 | (73.4) |

| | |
|---|---|
| 95% CI for Concordant [1] | (62.3, 82.7) |

[1] 95% exact confidence interval is estimated by Clopper-Pearson method, Clopper-Pearson Exact CI.

The duration of response data (a key secondary endpoint) is shown in Table 11.

TABLE 11
Key Secondary Endpoint: DOR

| DOR_{INV} | Total (N = 41) | |
|---|---|---|
| Number of responders | 41 | |
| Number of patients with events, n | 26 | (63.4) |
| (%) | | |
| PD | 25 | (61.0) |
| Death | 1 | (2.4) |
| Number of patients censored, n (%) | 15 | (36.6) |
| Start of new anti-cancer therapy | 3 | (7.3) |
| PD or death after missing ≥2 | 1 | (2.4) |
| consecutive radiological | | |
| assessments | | |
| Data cutoff | 11 | (26.8) |
| Estimated duration of response | | |
| (months) | | |
| Median (95% CI) | 8.25 | (5.55, 10.78) |
| 25th Percentile (95% CI) | 4.63 | (4.21, 6.70) |
| 75th Percentile (95% CI) | 12.62 | (9.07, —) |

| | |
|---|---|
| Min, Max | 0.99+, 16.89+ |

Progression free response (a secondary endpoint) data is shown in Table 12.

TABLE 12
Secondary Endpoint: PFS

| | | Total (N = 79) | |
|---|---|---|---|
| Number of patients with events, n (%) | | 62 | (78.5) |
| PD | | 58 | (73.4) |
| Death | | 4 | (5.1) |
| Number of patients censored, n (%) | | 17 | (21.5) |
| No post-baseline radiological assessments, and | | 0 | |
| patient did not die within 105 days of first dose | | | |
| New anti-cancer therapy prior to PD or death | | 5 | (6.3) |
| No death or progression | | 11 | (13.9) |
| PD or death after missing 2 or more consecutive | | 1 | (1.3) |
| radiological assessments | | | |
| Estimated progression free survival time (months) | | | |
| Median (95% CI) | | 6.93 | (5.85, 9.59) |
| 25th Percentile (95% CI) | | 3.98 | (2.66, 5.72) |
| 75th Percentile (95% CI) | | 11.01 | (10.02, 13.67) |
| Min, Max | | 0.46, 19.58+ | |
| Estimated probabilities of PFS at, (95% CI) | | | |
| 3 | months | 0.80 | (0.69, 0.87) |
| 6 | months | 0.61 | (0.49, 0.71) |
| 9 | months | 0.39 | (0.28, 0.50) |
| 12 | months | 0.19 | (0.10, 0.29) |

The estimated overall survival data is shown in Table 13.

TABLE 13
Secondary Endpoint: OS (Estimated)

| | | Total (N = 79) | |
|---|---|---|---|
| Number of patients with death events, n (%) | | 15 | (19.0) |
| Number of patients censored, n (%) | | 64 | (81.0) |
| Estimated overall survival time (months) | | | |
| Median (95% CI) | | — | (19.19, —) |
| 25th Percentile (95% CI) | | 17.28 | (14.92, —) |
| 75th Percentile (95% CI) | | — | (—, —) |
| Min, Max | | 1.38, 23.20+ | |
| Estimated probabilities of OS at, (95% CI) | | | |
| 3 | months | 0.97 | (0.90, 0.99) |
| 6 | months | 0.96 | (0.89, 0.99) |
| 9 | months | 0.90 | (0.81, 0.95) |
| 12 | months | 0.89 | (0.79, 0.94) |
| 15 | months | 0.84 | (0.72, 0.91) |
| RMST for OS at, (95% CI) | | | |
| 3 | months | 2.96 | (2.89, 3.04) |
| 6 | months | 5.85 | (5.64, 6.06) |
| 9 | months | 8.68 | (8.36, 9.00) |
| 12 | months | 11.35 | (10.86, 11.84) |
| 15 | months | 13.98 | (13.30, 14.66) |

The CA-125 response rate for the response-evaluable population (47 patients) is shown in Tables 14A and B.

TABLE 14A
Secondary Endpoint: CA-125 Response Rate

| | Total (N = 47) |
|---|---|
| CA-125 Response Rate, n (%) | 35 (74.5) |
| 95% Exact CI [2] | (59.7, 86.1) |

[2] 95% exact confidence interval is estimated by Clopper-Pearson method, Clopper-Pearson Exact CI.

TABLE 14B
Secondary Endpoint: CA-125

| | | | CA-125 Response Rate | | | |
|---|---|---|---|---|---|---|
| Clinical Parameter | Subgroup | | N | N (%) | | 95% CI [2] |
| All | — | | 47 | 35 | (74.5) | (59.7, 86.1) |
| Number of prior lines | 1 | | 1 | 0 | | (2.5, 100.0) |
| of therapy | | | | | | |
| | 2 | | 28 | 22 | (78.6) | (59.0, 91.7) |
| | 3 | | 14 | 10 | (71.4) | (41.9, 91.6) |
| | 4+ | | 4 | 3 | (75.0) | (19.4, 99.4) |
| | 1 or 2 | | 29 | 22 | (75.9) | (56.5, 89.7) |
| Prior exposure to | Yes | | 36 | 26 | (72.2) | (54.8, 85.8) |
| PARPi | | | | | | |
| | No | | 9 | 8 | (88.9) | (51.8, 99.7) |
| | Uncertain [3] | | 2 | 1 | (50.0) | (1.3, 98.7) |
| Prior exposure to | Yes | | 30 | 22 | (73.3) | (54.1, 87.7) |
| Bevacizumab | | | | | | |
| | No | | 17 | 13 | (76.5) | (50.1, 93.2) |
| Most recent platinum- | ≤12 | months | 27 | 17 | (63.0) | (42.4, 80.6) |
| free interval | | | | | | |
| | >12 | months | 18 | 16 | (88.9) | (65.3, 98.6) |

| | | | | |
|---|---|---|---|---|
| Missing | 2 | 2 | (100.0) | (15.8, 100.0) |

The overall response rate in various patient subsets are shown in Table 15.

TABLE 15
ORR in Subset Analysis: Prior LoT, Prior PARPi, Prior Bev, PFI.

Clinical

| Parameter | Subgroup | | N | ORR, n (%) [1] | | 95% CI [2] |
|---|---|---|---|---|---|---|
| All | — | | 79 | 41 | (51.9) | 40.4, 63.3 |
| Number of | 1 | | 1 | 0 | | 0.0, 97.5 |
| prior lines | | | | | | |
| of therapy | | | | | | |
| | 2 | | 48 | 27 | (56.3) | 41.2, 70.5 |
| | 3 | | 24 | 12 | (50.0) | 29.1, 70.9 |
| | 4+ | | 6 | 2 | (33.3) | 4.3, 77.7 |
| | 1 or 2 | | 49 | 27 | (55.1) | 40.2, 69.3 |
| Prior exposure | Yes | | 64 | 30 | (46.9) | 34.3, 59.8 |
| to PARPi | | | | | | |
| | Progression [3] | | 59 | 27 | (45.8) | 32.7, 59.2 |
| | No Progression [3] | | 5 | 3 | (60.0) | 14.7, 94.7 |
| | No | | 12 | 9 | (75.0) | 42.8, 94.5 |
| | Uncertain [4] | | 3 | 2 | (66.7) | 9.4, 99.2 |
| Prior exposure | Yes | | 51 | 25 | (49.0) | 34.8, 63.4 |
| to | | | | | | |
| Bevacizumab | | | | | | |
| | No | | 28 | 16 | (57.1) | 37.2, 75.5 |
| Most recent | ≤12 | months | 43 | 18 | (41.9) | 27.0, 57.9 |
| platinum-free | | | | | | |
| interval | | | | | | |
| | >12 | months | 34 | 22 | (64.7) | 46.5, 80.3 |

| | | | | |
|---|---|---|---|---|
| Missing | 2 | 1 | (50.0) | 1.3, 98.7 |

INV = Investigator;
CI = confidence interval;
[1] Objective response rate is defined as proportion of patients with a confirmed best overall response of complete or partial response.
[2] 95% exact confidence interval is estimated by Clopper-Pearson method, Clopper-Pearson Exact CI.
[3] If the patient had progression within 30 days after the last dosing of PARP inhibitor or progression is listed as the reason for treatment discontinuation of PARP inhibitor, the patient will be defined as progression on prior PARP inhibitor. If neither, the patient will be defined as no progression.
[4] For patients who participated in double blind trials evaluating PARPi vs placebo and the actual treatment was not known.

The duration of response in various patient subsets are shown in Table 16.

TABLE 16
Duration of response (DOR) in participant subgroups calculated
among participants who had a complete or partial response.

| | | DOR | Median DOR | |
|---|---|---|---|---|
| Subgroup | | subgroup n | months [95% CI] | |
| Prior lines of therapy | | | | |
| 1 or 2 | | 27 | 7.44 | [4.63-9.66] |
| 3 | | 12 | 8.41 | [4.63-NR] |
| ≥4 | | 2 | NR | [2.69-NR] |
| Exposure to PARPis | | | | |
| Yes | | 30 | 8.25 | [5.45-10.78] |
| Progression on PARPi ^{a} | | 27 | 7.33 | [5.03-10.78] |
| Without progression on PARPi | | 3 | 8.41 | [6.97-NR] |
| No | | 9 | 8.77 | [3.52-NR] |
| Unknown | | 2 | 4.21 | [NR-NR] |
| Exposure to bevacizumab | | | | |
| Yes | | 25 | 8.41 | [4.63-NR] |
| No | | 16 | 7.01 | [4.40-NR] |
| Most recent PFI^{b} | | | | |
| ≤12 | months | 18 | 7.33 | [4.27-10.78] |
| >12 | months | 22 | 8.33 | [4.63-NR] |

| | | |
|---|---|---|
| Missing | 1 | NR |

^{a} If the participant had progression of disease within 30 days after the last dosing of a PARPi or progression was listed as the reason for treatment discontinuation of a PARPi, the participant was defined as having progressive disease on prior PARPi and was included in this category.
^{b}Platinum-free interval is defined as time from last dose of the latest line platinum therapy to the date of disease progression and/or relapse (recurrence) following that line of therapy (time rounded to whole number).

An overview of treatment emergent adverse events (safety data) is shown in Tables 17A-G.

TABLE 17A
Overview of Treatment Emergent Adverse
Events (TEAE) Safety Population

| Number of Patients with Events, n (%) | Total (N = 79) | |
|---|---|---|
| Treatment-Emergent Adverse Event | 78 | (99) |
| Maximum Grade 3+ | 40 | (51) |
| Maximum Grade 3 | 37 | (47) |
| Maximum Grade 4 | 1 | (1) |
| Maximum Grade 5 | 2 | (3) |
| Serious TEAE | 15 | (19) |
| TEAE Leading to Dose Modification in Study Treatment | 52 | (66) |
| TEAE Leading to Dose Reduction in Study Treatment | 33 | (42) |
| TEAE Leading to Dose Delay/held in Study Treatment | 48 | (61) |
| TEAE Leading to Study Treatment Discontinuation | 13 | (16) |
| TEAE Leading to Death | 2 | (3) |

TABLE 17B
TEAE in >15%

| Preferred Term, n (%) | Total (N = 79) |
|---|---|
| Number of events, m | 1070 |
| Patients with any event, n (%) | 78 (99) |
| Vision blurred | 50 (63) |
| Dry eye | 29 (37) |
| Nausea | 29 (37) |
| Keratopathy | 26 (33) |
| Diarrhoea | 24 (30) |
| Asthenia | 23 (29) |
| Neuropathy peripheral | 22 (28) |
| Cataract | 19 (24) |
| Arthralgia | 16 (20) |
| Aspartate aminotransferase increased | 16 (20) |
| Vomiting | 15 (19) |
| Alanine aminotransferase increased | 14 (18) |
| Fatigue | 14 (18) |
| Constipation | 13 (16) |
| Headache | 13 (16) |
| COVID-19 | 12 (15) |
| Photophobia | 12 (15) |

TABLE 17C
TEAE Summary: Ocular >15%

| System Organ Class, n (%) | Total |
|---|---|
| Preferred Term, n (%) | (N = 79) |
| Number of events, m | 1070 |
| Patients with any TEAE, n (%) | 78 (99) |
| Eye disorders | 64 (81) |
| Vision blurred | 50 (63) |
| Dry eye | 29 (37) |
| Keratopathy | 26 (33) |
| Cataract | 19 (24) |
| Photophobia | 12 (15) |
| Keratitis | 7 (9) |
| Visual acuity reduced | 7 (9) |
| Corneal epithelial microcysts | 5 (6) |
| Eye pain | 4 (5) |
| Ocular hyperaemia | 3 (4) |
| Foreign body sensation in eyes | 2 (3) |
| Punctate keratitis | 2 (3) |
| Visual impairment | 2 (3) |
| Vitreous floaters | 2 (3) |
| Abnormal sensation in eye | 1 (1) |
| Corneal disorder | 1 (1) |

TABLE 17D
TEAE Summary: GI, Nervous System, and General Disorders

| | | |
|---|---|---|
| Gastrointestinal disorders | 51 | (65) |
| Nausea | 29 | (37) |
| Diarrhoea | 24 | (30) |
| Vomiting | 15 | (19) |
| Constipation | 13 | (16) |
| Abdominal pain | 11 | (14) |
| Nervous system disorders | 51 | (65) |
| Neuropathy peripheral | 21 | (27) |
| Headache | 13 | (16) |
| Neurotoxicity | 10 | (13) |
| Dysgeusia | 9 | (11) |
| Peripheral sensory neuropathy | 7 | (9) |
| Paraesthesia | 4 | (5) |
| General disorders and administration site conditions | 44 | (56) |
| Asthenia | 23 | (29) |
| Fatigue | 14 | (18) |

TABLE 17E
Grade 3 TEAEs

| System Organ Class, n (%) | Total | |
|---|---|---|
| Preferred Term, n (%) | (N = 79) | |
| Number of events, m | 66 | |
| Patients with any TEAE, n (%) | 40 | (51) |
| Eye disorders | 17 | (22) |
| Vision blurred | 8 | (10) |
| Cataract | 6 | (8) |
| Keratopathy | 3 | (4) |
| Dry eye | 2 | (3) |
| Corneal epithelial microcysts | 1 | (1) |
| Eye pain | 1 | (1) |
| Keratitis | 1 | (1) |
| Visual acuity reduced | 1 | (1) |
| Nervous system disorders | 9 | (11) |
| Neuropathy peripheral | 3 | (4) |
| Neurotoxicity | 2 | (3) |
| Ataxia | 1 | (1) |
| Paraesthesia | 1 | (1) |
| Peripheral sensory neuropathy | 1 | (1) |
| Syncope | 1 | (1) |
| Respiratory, thoracic and mediastinal disorders | 4 | (5) |
| Pneumonitis | 3 | (4) |

TABLE 17F
Serious TEAEs

| Preferred Term, n (%) | Total (N = 79) |
|---|---|
| Number of events, m | 9 |
| Patients with any event, n (%) | 7 (9) |
| Pneumonitis | 3 (4) |
| Keratitis | 1 (1) |
| Nausea | 1 (1) |
| Neuropathy peripheral | 1 (1) |
| Vomiting | 1 (1) |

TABLE 17G
TEAEs Leading to Discontinuation

| Preferred Term, n (%) | Total (N = 79) |
|---|---|
| Number of events, m | 14 |
| Patients with any event, n (%) | 13 (16) |
| Pneumonitis | 3 (4) |
| Cataract | 2 (3) |
| Corneal epithelial microcysts | 2 (3) |
| Neuropathy peripheral | 2 (3) |
| Vision blurred | 2 (3) |
| Cystitis | 1 (1) |
| Interstitial lung disease | 1 (1) |
| Keratopathy | 1 (1) |

The phase 2 PICCOLO trial described herein evaluated the efficacy and safety of mirvetuximab soravtansine in third-line and later PSOC. The trial built upon the clinical benefit observed with mirvetuximiab soravtansine in other populations of patients with ovarian cancer and further demonstrated that mirvetuximab soravtansine is efficacious in a variety of patients with FRα-positive ovarian cancer. The PICCOLO trial met its primary endpoint, with an investigator-assessed ORR of 51.9% (95% CI, 40.4-63.3) in the overall population, including a 7.6% CR rate and 44.3% PR rate. These responses were durable, as demonstrated by the key secondary endpoint of DOR (median DOR, 8.25 months [95% CI, 5.55-10.78]), and further supported by the investigator-assessed median PFS of 6.93 months (95% CI, 5.85-9.59). Additionally, 11 participants were still on treatment at time of data cutoff. The safety profile of mirvetuximab soravtansine was consistent with that of previous trials, and no new safety signals were observed. Moore et al., N Engl J Med 389:2162-2174, 2023; Matulonis et al., J Clin Oncol 41:2436-2445, 2023. Furthermore, 43.5% of screened participants demonstrated positive FRα expression, underscoring the utility of mirvetuximab soravtansine as a viable treatment option in a substantial proportion of patients with PSOC.

The efficacy benefits seen among subgroups in PICCOLO suggest that mirvetuximab soravtansine may be an effective treatment option for patients with recurrent PSOC who are heavily pretreated and have experienced disease progression with other treatments. This includes participants with 3 prior lines of therapy (ORR, 50.0% [95% CI, 29.1-70.9]), prior bevacizumab treatment (ORR, 49.0% [95% CI, 34.8-63.4]), and prior PARPi treatment (46.9% [95% CI, 34.3-59.8]). Further, a large proportion of participants in the PICCOLO trial (74.7%, n=59) had prior documented PD while on or within 30 days of PARPi treatment; these participants demonstrated an ORR of 45.8% [95% CI, 32.7-59.2]) with mirvetuximab soravtansine. This response is notable given the significant use of PARPi therapy in EOC and the few efficacious and tolerable treatment options available after PD with PARPi. O'Malley et al., Target Oncol 18:471-503, 2023; in Lele S (ed): Ovarian Cancer. Brisbane (AU), 2022.

Treatment for later-line PSOC following prior PARPi typically consists of retreatment with platinum-based chemotherapy with or without bevacizumab, followed by bevacizumab as maintenance therapy. O'Malley et al., Target Oncol 18:471-503, 2023. Diminished efficacy with subsequent therapies (such as platinum-based chemotherapy re-treatment) following disease progression on PARPi treatment has been observed. Frenel et al., Ann Oncol 33:1021-1028, 2022; Cecere et al., Gynecol Oncol 156:38-44, 2020; Romeo et al., Cancers (Basel) 14, 2022; Harter et al. Presented at: 2023 American Society of Clinical Oncology; Jun. 2-6, 2023; Chicago, IL. Abstract 5550; Rose et al., Anticancer Drugs 32:1086-1092, 2021; Park et al., Gynecol Oncol 165:97-104, 2022. Post hoc analysis of the SOLO-2 trial found median time to second progression (TTSP) on subsequent chemotherapy was significantly longer in patients who previously received placebo versus PARPi (12.1 months with placebo versus 6.9 months with olaparib; HR 2.17, [95% CI, 1.47-3.19]). Similar findings were observed in post hoc analyses of the PAOLA-1 study; time from first subsequent therapy to second subsequent therapy was shortest in patients who progressed during PARPi maintenance therapy (6.1 months), compared with those who progressed afterwards (11.4 months) or who were treated with bevacizumab plus placebo (11.9 months). Further, a retrospective real-world analysis of patients who received platinum-based chemotherapy with or without bevacizumab following disease progression after PARPi found ORR was 41.9%, median PFS was 6.6 months (95% CI, 6.0-9.2), and median OS was 20.6 months (95% CI, 13.6-28.9). Other retrospective analyses, real-world data, and meta-analyses of PARPi-treated patients have corroborated these findings and highlighted the unmet need in this population.

No new mirvetuximab soravtansine safety signals were observed in the PICCOLO trial, as the most common TEAEs were generally low-grade gastrointestinal, neurosensory, and resolvable ocular events. Overall, grade ≥3 TEAEs occurred in 51% of participants (including 1 grade 4 and 2 grade 5 events), and grade ≥3 TRAEs occurred in 35% of participants. Pneumonitis, an AE of concern for ADCs, occurred in 8 participants (10%), and peripheral neuropathy, a common issue with tubulin-targeting agents, occurred in 22 participants (28%). In the PICCOLO trial, TEAEs resulted in mirvetuximab soravtansine discontinuation in 13 participants (16%), which may be attributed to the increased treatment options for patients with PSOC and longer median time on therapy, with a median of 10 cycles (range 2-21) of mirvetuximab soravtansine administered prior to discontinuation due to toxicity.

While definitive conclusions cannot be made from a single-arm trial without a comparator arm, PICCOLO demonstrated notable efficacy in a heavily pretreated population of participants with PSOC, including 81% of participants who had prior PARPi therapy. The generalizability of these results may be limited by the lack of racial and ethnic diversity among trial participants and the FRα eligibility criteria.

After 2+ lines of platinum therapy, additional platinum-based therapy for patients with PSOC carries the potential for significant cumulative toxicity and decline with each subsequent line of treatment. The clinical trial described herein shows that mirvetuximab soravtansine is a safe and efficacious treatment in patients with recurrent platinum-sensitive, high-grade serous epithelial ovarian cancer (EOC), primary peritoneal, or fallopian tube cancer, whose tumors express a high-level of FRα, in patients with at least two prior lines of therapy. Mirvetuximab soravtansine is the first ADC (antibody-drug conjugate) for ovarian cancer with proven efficacy and it is the only FDA-approved biomarker-directed therapy for platinum-resistant ovarian cancer. These data position mirvetuximab soravtansine as a new standard of care for patients with recurrent platinum-sensitive ovarian cancer (rPSOC) and high folate receptor alpha (FRα) expression.

It is to be appreciated that the Detailed Description section, and not the Summary and Abstract sections, is intended to be used to interpret the claims. The Summary and Abstract sections sets forth one or more, but not all, exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects provided herein will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects provided herein, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

1. A method for treating a folate receptor alpha (FRα)-expressing recurrent platinum-sensitive ovarian cancer (rPSOC) in a human patient in need thereof, comprising intravenously administering once every three weeks to the patient a therapeutically effective amount of mirvetuximab soravtansine,
wherein the therapeutically effective amount is 6 mg/kg of adjusted ideal body weight (AIBW) of the patient.

2. A method for treating a folate receptor alpha (FRα)-expressing recurrent platinum-sensitive ovarian cancer (rPSOC) in a human patient in need thereof, comprising intravenously administering once every three weeks to the patient a therapeutically amount of mirvetuximab soravtansine, wherein the therapeutically effective amount is 6 mg/kg of adjusted ideal body weight (AIBW) of the patient, and wherein
the administration increases the human patient's overall survival,
the administration results in a decrease in tumor size,
the administration results in a decrease in cancer antigen 125 (CA-125) expression,
the administration results in an objective response rate (ORR) of about greater than 25%,
wherein the administration results in the human patient achieving a duration of response (DOR) of at least 4 months,
the administration results in the human patient achieving a progression-free survival (PFS) of at least 3.98 months, and/or
the administration results in an about 80% chance of the human patient achieving a progression-free survival (PFS) of at least 3 months.

3. The method of claim 2, wherein the rPSOC is epithelial ovarian cancer (EOC), high-grade serous EOC, primary peritoneal cancer or fallopian tube cancer.

4-6. (canceled)

7. The method of claim 2, wherein the patient has had at least one line of platinum-containing therapy prior to initiation of the present method.

8. The method of claim 7, wherein the patient has had a platinum-free interval of
at least 6 months prior to initiation of the present method,
between 6 months and 12 months prior to initiation of the present method, or
more than 12 months prior to initiation of the present method.

9-10. (canceled)

11. The method of claim 2, wherein the patient has had one independent non-platinum cytotoxic therapy prior to initiation of the present method.

12. The method of claim 2, wherein the patient has a platinum allergy.

13. The method of claim 2, wherein the patient has had at least two lines of therapy prior to initiation of the present method.

14. The method of claim 2, wherein the mirvetuximab soravtansine is administered at a rate of 1 mg/min, 3 mg/ml, or 5 mg/ml.

15-16. (canceled)

17. The method of claim 2, wherein the mirvetuximab soravtansine is administered as a monotherapy.

18. The method of claim 2, wherein a cancer sample from the patient exhibits increased expression of FRα measured by an immunohistochemistry (IHC) assay that can determine the percentage of cancer cells with moderate (2+) and/or strong (3+) membrane staining compared to the total number of viable tumor cells.

19. (canceled)

20. The method of claim 18, wherein
at least 25% of cells in a cancer sample obtained from the patient have an immunohistochemistry (IHC) score of at least 2,
at least 75% of cells in a cancer sample obtained from the patient have an immunohistochemistry (IHC) score of at least 2,
a positive FRα expression score is defined as ≥75% of viable tumor cells with moderate (2+) and/or strong (3+) membrane staining,
a negative FRα expression is defined as <75% of viable tumor cells with moderate (2+) and/or strong (3+) membrane staining, and/or
the patient's FRα-expressing cancer sample displays a positive FRα expression score as defined by the assay.

21-24. (canceled)

25. The method of claim 2, wherein the subject has at least one measurable disease lesion as defined by RECIST v1.1.

26. The method of claim 2, wherein the patient has a BRCA mutation.

27. The method of claim 2, wherein the patient does not have a BRCA mutation.

28. The method of claim 2, wherein the patient has had PARPi therapy prior to initiation of the present method.

29. The method of claim 28, wherein the patient has had disease progression (PD) while receiving PARPi therapy or within 30 days of receiving PARPi prior to initiation of the present method.

30. The method of claim 28, wherein the patient has had no disease progression (PD) while receiving PARPi therapy or within 30 days of receiving PARPi prior to initiation of the present method.

31. The method of claim 2, wherein the patient has had bevacizumab therapy and/or taxane therapy prior to initiation of the present method.

32. (canceled)

33. The method of claim 2, further comprising administering a corticosteroid eye drop and/or artificial tears to the patient.

34-35. (canceled)


SEQUENCE LISTING (26 sequences)

SEQ ID NO: 1 - protein, 257 aa, Homo sapiens
MAQRMTTQLLLLLVWVAVVGEAQTRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 2 - skipped

SEQ ID NO: 3 - protein, 5 aa, synthetic construct
GYFMN

SEQ ID NO: 4 - protein, 17 aa, synthetic construct
RIHPYDGDTFYNQKFQG

SEQ ID NO: 5 - protein, 9 aa, synthetic construct
YDGSRAMDY

SEQ ID NO: 6 - protein, 15 aa, synthetic construct
KASQSVSFAGTSLMH

SEQ ID NO: 7 - protein, 7 aa, synthetic construct
RASNLEA

SEQ ID NO: 8 - protein, 9 aa, synthetic construct
QQSREYPYT

SEQ ID NO: 9 - protein, 10 aa, synthetic construct
GYTFTGYFMN

SEQ ID NO: 10 - protein, 10 aa, synthetic construct
RIHPYDGDTF

SEQ ID NO: 11 - protein, 9 aa, synthetic construct
YDGSRAMDY

SEQ ID NO: 12 - protein, 17 aa, synthetic construct
RIHPYDGDTFYNQNFKD

SEQ ID NO: 13 - protein, 5 aa, synthetic construct
NSYIH

SEQ ID NO: 14 - protein, 17 aa, synthetic construct
WIYPESLNTQYNEKFKA

SEQ ID NO: 15 - protein, 13 aa, synthetic construct
RGIYYYSPYALDH

SEQ ID NO: 16 - protein, 16 aa, synthetic construct
KSSKSLLNSDGFTYLD

SEQ ID NO: 17 - protein, 7 aa, synthetic construct
LVSNHFS

SEQ ID NO: 18 - protein, 9 aa, synthetic construct
FQSNYLPLT

SEQ ID NO: 19 - protein, 118 aa, synthetic construct
QVQLVQSGAEVVKPGASVKISCKASGYTFTGYFMNWVKQSPGQSLEWIGRIHPYDGDTFYNQKFQGKATLTVDKSSNTAHMELLSLTSEDFAVYYCTRYDGSRAMDYWGQGTTVTVSS

SEQ ID NO: 20 - protein, 112 aa, synthetic construct
DIVLTQSPLSLAVSLGQPAIISCKASQSVSFAGTSLMHWYHQKPGQQPRLLIYRASNLEAGVPDRFSGSGSKTDFTLTISPVEAEDAATYYCQQSREYPYTFGGGTKLEIKR

SEQ ID NO: 21 - protein, 122 aa, synthetic construct
QVQLQQSGPELVKPGASVRISCKASGYTFTNSYIHWVKKRPGQGLEWIGWIYPESLNTQYNEKFKAKATLTADKSSSTSYMQLSSLTSEDSAVYFCARRGIYYYSPYALDHWGQGASVTVSS

SEQ ID NO: 22 - protein, 114 aa, synthetic construct
SDVVLTQTPLSLPVNIGDQASISCKSSKSLLNSDGFTYLDWYLQKPGQSPQLLIYLVSNHFSGVPDRFSGSGSGTDFTLKISRVEAEDLGVYYCFQSNYLPLTFGGGTKLEIKR

SEQ ID NO: 23 - protein, 447 aa, synthetic construct
QVQLVQSGAEVVKPGASVKISCKASGYTFTGYFMNWVKQSPGQSLEWIGRIHPYDGDTFYNQKFQGKATLTVDKSSNTAHMELLSLTSEDFAVYYCTRYDGSRAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 24 - protein, 446 aa, synthetic construct
QVQLQQSGPELVKPGASVRISCKASGYTFTNSYIHWVKKRPGQGLEWIGWIYPESLNTQYNEKFKAKATLTADKSSSTSYMQLSSLTSEDSAVYFCARRGIYYYSPYALDHWGQGASVTVSSAKTTPPSVYPLAPGSAAQTNSMVTLGCLVKGYFPEPVTVTWNSGSLSSGVHTFPAVLESDLYTLSSSVTVPSSMRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 25 - protein, 218 aa, synthetic construct
DIVLTQSPLSLAVSLGQPAIISCKASQSVSFAGTSLMHWYHQKPGQQPRLLIYRASNLEAGVPDRFSGSGSKTDFTLTISPVEAEDAATYYCQQSREYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 220 aa, synthetic construct
SDVVLTQTPLSLPVNIGDQASISCKSSKSLLNSDGFTYLDWYLQKPGQSPQLLIYLVSNHFSGVPDRFSGSGSGTDFTLKISRVEAEDLGVYYCFQSNYLPLTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC